(19) United States
(12) Patent Application Publication
(10) Pub. No.: US 2004/0101843 A1
(43) Pub. Date: May 27, 2004

(54) Detection of methylated DNA sites

(76) Inventor: Gerald Zon, San Carlos, CA (US)

Correspondence Address: FINNEGAN, HENDERSON, FARABOW, GARRETT & DUNNER, LLP, 1300 I STREET, NW, WASHINGTON, DC, 20005, US

(21) Appl. No.: 10/302,299

(22) Filed: Nov. 22, 2002

(51) Int. Cl. C12Q 1/68; C12P 19/34

(52) U.S. Cl. 435/6; 435/91.2

(57) ABSTRACT

The present invention relates to methods and kits for determining the methylation state of target cytosines in a sample using ligation.

### I. FIELD OF THE INVENTION

The present invention generally relates to methods and materials for the detection of methylation of DNA.

### II. BACKGROUND OF THE INVENTION

Assessment of methylation of DNA is useful in many research, diagnostic, medical, forensic and industrial fields.

In certain instances, methylation has regulatory effects on gene expression and may play an important role in a variety of settings, including gene inactivation, cell differentiation, tumor growth, X-chromosome inactivation, and genomic imprinting. For example, in certain instances, extensive methylation in a promoter region has been shown to suppress transcription. Thus, in certain instances, methylation may play a role in developmental gene regulation and cell differentiation.

Also, aberrant methylation has been described in several tumors and immortalized and transformed cells. Hypermethylation of tumor suppressor regions has been associated with human cancers. Thus, in certain instances, determination of methylation may be useful in tumor assessment.

In certain instances, determining methylation may be used to study gene regulation and may serve as a marker for various disease states and may be useful for tissue typing. Determining methylation in certain instances may be useful for identifying individuals (i.e. fingerprinting) or for other industrial applications.

### III. SUMMARY OF THE INVENTION

In certain embodiments, the invention comprises methods for determining the methylation state of a target cytosine in at least one target nucleic acid sequence in a sample.

In certain embodiments, the method comprises forming a ligation reaction composition comprising the sample and a ligation probe set for each target nucleic acid sequence, the probe set comprising (a) at least one first probe, comprising a target-specific portion and (b) at least one second probe, comprising a target-specific portion; subjecting the ligation reaction composition to at least one cycle of ligation, under conditions effective to ligate together first and second probes that are hybridized adjacent to one another on the target nucleic acid sequence only if the target cytosine is methylated or only if the target cytosine is unmethylated, to form a ligation product; and detecting the presence or absence of the ligation product to determine the methylation state of the target cytosine.

In certain embodiments, the invention comprises a method for determining the methylation state of a target cytosine in at least one target nucleic acid sequence in a sample, comprising: forming a test composition by incubating the at least one target nucleic acid sequence with a modifying agent that converts unmethylated cytosine to a converted nucleotide, but does not convert methylated cytosine to the converted nucleotide, to obtain at least one test target nucleic acid sequence; forming a ligation reaction composition comprising at least a portion of the test composition and a ligation probe set for each target nucleic acid sequence, the probe set comprising (a) at least one first probe, comprising a target-specific portion and (b) at least one second probe, comprising a target-specific portion, wherein the probes in each probe set are suitable for ligation together when hybridized adjacent to one another on a complementary test target nucleic acid sequence, and wherein at least one of the at least one first probe and the at least one second probe of each probe set comprises a test nucleotide that aligns opposite the target cytosine or the converted nucleotide if the probe is hybridized to the test target nucleic acid sequence, wherein the test nucleotide is complementary to cytosine or to the converted nucleotide; subjecting the ligation reaction composition to at least one cycle of ligation, wherein adjacently hybridizing probes are ligated together to form a ligation product; and detecting the presence or absence of the ligation product to determine the methylation state of the target cytosine.

In certain embodiments, the invention comprises a method for determining the methylation state of a target cytosine in at least one target nucleic acid sequence in a sample, comprising: forming a test composition by incubating the at least one target nucleic acid sequence with a modifying agent that modifies unmethylated cytosine, but does not modify methylated cytosine, to obtain at least one test target nucleic acid sequence; forming a ligation reaction composition comprising at least a portion of the test composition and a ligation probe set for each target nucleic acid sequence, the probe set comprising (a) at least one first probe, comprising a target-specific portion and (b) at least one second probe, comprising a target-specific portion; subjecting the ligation reaction composition to at least one cycle of ligation, under conditions effective to ligate together first and second probes that are hybridized adjacent to one another on the test target nucleic acid sequence only if the target cytosine is modified or only if the target cytosine is unmodified, to form a ligation product; and detecting the presence or absence of the ligation product to determine the methylation state of the target cytosine.

In certain embodiments, the invention comprises a method for determining the methylation state of a target cytosine in at least one target nucleic acid sequence in a sample, comprising: forming a test composition by incubating the at least one target nucleic acid sequence with a modifying agent that modifies methylated cytosine, but does not modify unmethylated cytosine, to obtain at least one test target nucleic acid sequence; forming a ligation reaction composition comprising at least a portion of the test composition and a ligation probe set for each target nucleic acid sequence, the probe set comprising (a) at least one first probe, comprising a target-specific portion and (b) at least one second probe, comprising a target-specific portion; subjecting the ligation reaction composition to at least one cycle of ligation, under conditions effective to ligate together first and second probes that are hybridized adjacent to one another on the test target nucleic acid sequence only if the target cytosine is modified or only if the target cytosine is unmodified, to form a ligation product; and detecting the presence or absence of the ligation product to determine the methylation state of the target cytosine.

In certain embodiments, the invention comprises a method for determining the methylation state of a target cytosine in at least one target nucleic acid sequence in a sample, comprising: forming a ligation reaction composition comprising the sample and a ligation probe set for each target nucleic acid sequence, the probe set comprising (a) at least one first probe, comprising a target-specific portion and a 5′ primer-specific portion, wherein the 5′ primer-specific portion comprises a sequence, and (b) at least one second probe, comprising a target-specific portion and a 3′ primer-specific portion, wherein the 3′ primer-specific portion comprises a sequence; forming an amplification test composition by subjecting the ligation reaction composition to at least one cycle of ligation, under conditions effective to ligate together first and second probes that are hybridized adjacent to one another on the target nucleic acid sequence only if the target cytosine is methylated or only if the target cytosine is unmethylated, to form a ligation product; forming an amplification reaction composition comprising: at least a portion of the amplification test composition; a polymerase; and at least one primer set, the primer set comprising (i) at least one first primer comprising the sequence of the 5′ primer-specific portion of the ligation product, and (ii) at least one second primer comprising a sequence complementary to the sequence of the 3′ primer-specific portion of the ligation product; subjecting the amplification reaction composition to at least one amplification reaction; and determining the methylation state of the target cytosine by detecting the presence or absence of the ligation product.

In certain embodiments, the invention comprises a method for determining the methylation state of a target cytosine in at least one target nucleic acid sequence in a sample, comprising: forming a test composition by incubating the at least one target nucleic acid sequence with a modifying agent that converts unmethylated cytosine to a converted nucleotide, but does not convert methylated cytosine to the converted nucleotide, to obtain at least one test target nucleic acid sequence; forming a ligation reaction composition comprising at least a portion of the test composition and a ligation probe set for each target nucleic acid sequence, the probe set comprising (a) at least one first probe, comprising a target-specific portion and a 5′ primer-specific portion, wherein the 5′ primer-specific portion comprises a sequence, and (b) at least one second probe, comprising a target-specific portion and a 3′ primer-specific portion, wherein the 3′ primer-specific portion comprises a sequence, wherein the probes in each probe set are suitable for ligation together when hybridized adjacent to one another on a complementary test target nucleic acid sequence, and wherein at least one of the at least one first probe and the at least one second probe of each probe set comprises a test nucleotide that aligns opposite the target cytosine or the converted nucleotide if the probe is hybridized to the test target nucleic acid sequence, wherein the test nucleotide is complementary to cytosine or to the converted nucleotide; forming an amplification test composition by subjecting the ligation reaction composition to at least one cycle of ligation, wherein adjacently hybridizing probes are ligated together to form a ligation product; forming an amplification reaction composition comprising: at least a portion of the amplification test composition; a polymerase; and at least one primer set, the primer set comprising (i) at least one first primer comprising the sequence of the 5′ primer-specific portion of the ligation product, and (ii) at least one second primer comprising a sequence complementary to the sequence of the 3′ primer-specific portion of the ligation product; subjecting the amplification reaction composition to at least one amplification reaction; and determining the methylation state of the target cytosine by detecting the presence or absence of the ligation product.

In certain embodiments, the invention comprises a method for determining the methylation state of a target cytosine in at least one target nucleic acid sequence in a sample, comprising: forming a test composition by incubating the at least one target nucleic acid sequence with a modifying agent that modifies unmethylated cytosine, but does not modify methylated cytosine, to obtain at least one test target nucleic acid sequence; forming a ligation reaction composition comprising at least a portion of the test composition and a ligation probe set for each target nucleic acid sequence, the probe set comprising (a) at least one first probe, comprising a target-specific portion and a 5′ primer-specific portion, wherein the 5′ primer-specific portion comprises a sequence, and (b) at least one second probe, comprising a target-specific portion and a 3′ primer-specific portion; forming an amplification test composition by subjecting the ligation reaction composition to at least one cycle of ligation, under conditions effective to ligate together first and second probes that are hybridized adjacent to one another on the test target nucleic acid sequence only if the target cytosine is modified or only if the target cytosine is unmodified, to form a ligation product; forming an amplification reaction composition comprising: at least a portion of the amplification test composition; a polymerase; and at least one primer set, the primer set comprising (i) at least one first primer comprising the sequence of the 5′ primer-specific portion of the ligation product, and (ii) at least one second primer comprising a sequence complementary to the sequence of the 3′ primer-specific portion of the ligation product; subjecting the amplification reaction composition to at least one amplification reaction; and detecting the presence or absence of the ligation product to determine the methylation state of the target cytosine.

In certain embodiments, the invention comprises a method for determining the methylation state of a target cytosine in at least one target nucleic acid sequence in a sample, comprising: forming a test composition by incubating the at least one target nucleic acid sequence with a modifying agent that modifies methylated cytosine,.but does not modify unmethylated cytosine, to obtain at least one test target nucleic acid sequence; forming a ligation reaction composition comprising at least a portion of the test composition and a ligation probe set for each target nucleic acid sequence, the probe set comprising (a) at least one first probe, comprising a target-specific portion and a 5′ primer-specific portion, wherein the 5′ primer-specific portion comprises a sequence, and (b) at least one second probe, comprising a target-specific portion and a 3′ primer-specific portion; forming an amplification test composition by subjecting the ligation reaction composition to at least one cycle of ligation, under conditions effective to ligate together first and second probes that are hybridized adjacent to one another on the test target nucleic acid sequence only if the target cytosine is modified or only if the target cytosine is unmodified, to form a ligation product; forming an amplification reaction composition comprising: at least a portion of the amplification test composition; a polymerase; and at least one primer set, the primer set comprising (i) at least one first primer comprising the sequence of the 5′ primer-specific portion of the ligation product, and (ii) at least one second primer comprising a sequence complementary to the sequence of the 3′ primer-specific portion of the ligation product; subjecting the amplification reaction composition to at least one amplification reaction; and detecting the presence or absence of the ligation product to determine the methylation state of the target cytosine.

In certain embodiments kits for determining the methylation state of a target cytosine in at least one target nucleic acid sequence in a sample comprise: a modifying agent that modifies methylated cytosine, but does not modify unmethylated cytosine; and a ligation probe set for each target cytosine, the probe set comprising: (a) at least one first probe, comprising a target-specific portion, and (b) at least one second probe, comprising a target-specific portion, wherein the probes in each set are suitable for ligation together when hybridized adjacent to one another on a complementary target nucleic acid sequence.

In certain embodiments, kits for determining the methylation state of a target cytosine in at least one target nucleic acid sequence in a sample comprise: a modifying agent that modifies unmethylated cytosine, but does not modify methylated cytosine; and a ligation probe set for each target cytosine, the probe set comprising: (a) at least one first probe, comprising a target-specific portion, and (b) at least one second probe, comprising a target-specific portion, wherein the probes in each set are suitable for ligation together when hybridized adjacent to one another on a complementary target nucleic acid sequence.

In certain embodiments, kits for determining the methylation state of a target cytosine in at least one target nucleic acid sequence in a sample comprise: a ligation probe set for each target cytosine, the probe set comprising: (a) at least one first probe, comprising a target-specific portion, and (b) at least one second probe, comprising a target-specific portion, wherein the probes in each set are suitable for ligation together when hybridized adjacent to one another on a complementary target nucleic acid sequence; and a selective ligase that ligates together adjacently hybridized probes only if the target cytosine is methylated.

In certain embodiments, kits for determining the methylation state of a target cytosine in at least one target nucleic acid sequence in a sample comprise: a ligation probe set for each target cytosine, the probe set comprising: (a) at least one first probe, comprising a target-specific portion, and (b) at least one second probe, comprising a target-specific portion, wherein the probes in each set are suitable for ligation together when hybridized adjacent to one another on a complementary target nucleic acid sequence; and a selective ligase that ligates together adjacently hybridized probes only if the target cytosine is unmethylated.

### IV. BRIEF DESCRIPTION OF THE DRAWINGS

The skilled artisan will understand that the drawings, described below, are for illustration purposes only. The figures are not intended to limit the scope of the invention in any way.

FIGS. 1A to D. Schematic showing of ligaton probe sets according to certain exemplary embodiments.

FIG. 2. Schematic showing of certain embodiments comprising ligation and amplification.

FIG. 3 depicts certain embodiments comprising ligation.

FIG. 4 depicts certain embodiments comprising ligation and amplification.

FIG. 5 depicts certain embodiments comprising ligation.

### V. DETAILED DESCRIPTION OF CERTAIN EXEMPLARY EMBODIMENTS

It is to be understood that both the foregoing general description and the following detailed description are exemplary and explanatory only and are not restrictive of the invention, as claimed. In this application, the use of the singular includes the plural unless specifically stated otherwise. In this application, the use of “or” means “and/or” unless stated otherwise. Furthermore, the use of the term “including”, as well as other forms, such as “includes” and “included”, is not limiting. Also, terms such as “element” or “component” encompass both elements and components comprising one unit and elements and components that comprise more than one subunit unless specifically stated otherwise.

The section headings used herein are for organizational purposes only and are not to be construed as limiting the subject matter described. All documents, or portions of documents, cited in this application, including but not limited to patents, patent applications, articles, books, and treatises, are hereby expressly incorporated by reference in their entirety for any purpose. U.S. patent application Ser. Nos. 09/584,905, filed May 30, 2000, 09/724,755, filed Nov. 28, 2000, 10/011,993, filed Dec. 5, 2001, 60/412,225 filed Sep. 19, 2002, 60/421,035 filed Oct. 23, 2002, and Patent Cooperation Treaty Application No. PCT/US01/17329, filed May 30, 2001, are hereby expressly incorporated by reference in their entirety for any purpose.

### A. Definitions

The term “nucleotide base”, as used herein, refers to a substituted or unsubstituted aromatic ring or rings. In certain embodiments, the aromatic ring or rings contain at least one nitrogen atom. In certain embodiments, the nucleotide base is capable of forming Watson-Crick and/or Hoogsteen hydrogen bonds with an appropriately complementary nucleotide base. Exemplary nucleotide bases and analogs thereof include, but are not limited to, naturally occurring nucleotide bases adenine, guanine, cytosine, 6 methyl-cytosine, uracil, thymine, and analogs of the naturally occurring nucleotide bases, e.g., 7-deazaadenine, 7-deazaguanine, 7-deaza-8-azaguanine, 7-deaza-8-azaadenine, N6-Δ2-isopentenyladenine (6iA), N6-Δ2-isopentenyl-2-methylthioadenine (2ms6iA), N2-dimethylguanine (dmG), 7-methylguanine (7mG), inosine, nebularine, 2-aminopurine, 2-amino-6-chloropurine, 2,6-diaminopurine, hypoxanthine, pseudouridine, pseudocytosine, pseudoisocytosine, 5-propynylcytosine, isocytosine, isoguanine, 7-deazaguanine, 2-thiopyrimidine, 6-thioguanine, 4-thiothymine, 4-thiouracil, O⁶-methylguanine, N⁶-methyladenine, O⁴-methylthymine, 5,6-dihydrothymine, 5,6-dihydrouracil, pyrazolo[3,4-D]pyrimidines (see, e.g., U.S. Pat. Nos. 6,143,877 and 6,127,121 and PCT published application WO 01/38584), ethenoadenine, indoles such as nitroindole and 4-methylindole, and pyrroles such as nitropyrrole. Certain exemplary nucleotide bases can be found, e.g., in Fasman, 1989, Practical Handbook of Biochemistry and Molecular Biology, pp. 385-394, CRC Press, Boca Raton, Fla., and the references cited therein.

The term “nucleotide”, as used herein, refers to a compound comprising a nucleotide base linked to the C-1′ carbon of a sugar, such as ribose, arabinose, xylose, and pyranose, and sugar analogs thereof. The term nucleotide also encompasses nucleotide analogs. The sugar may be substituted or unsubstituted. Substituted ribose sugars include, but are not limited to, those riboses in which one or more of the carbon atoms, for example the 2′-carbon atom, is substituted with one or more of the same or different CI, F, —R, —OR, —NR₂ or halogen groups, where each R is independently H, C₁-C₆ alkyl or C₅-C₁₄ aryl. Exemplary riboses include, but are not limited to, 2′-(C1-C6)alkoxyribose, 2′-(C5 -C14)aryloxyribose, 2′,3′-didehydroribose, 2′-deoxy-3′-haloribose, 2′-deoxy-3′-fluororibose, 2′-deoxy-3′-chlororibose, 2′-deoxy-3′-aminoribose, 2′-deoxy-3′-(C1-C6)alkylribose, 2′-deoxy-3′-(C1 -C6)alkoxyribose and 2′-deoxy-3′-(C5-C14)aryloxyribose, ribose, 2′-deoxyribose, 2′,3′-dideoxyribose, 2′-haloribose, 2′-fluororibose, 2′-chlororibose, and 2′-alkylribose, e.g., 2′-O-methyl, 4′-α-anomeric nucleotides, 1′-α-anomeric nucleotides, 2′-4′- and 3′-4′-linked and other “locked” or “LNA”, bicyclic sugar modifications (see, e.g., PCT published application nos. WO 98/22489, WO 98/39352;, and WO 99/14226). Exemplary LNA sugar analogs within a polynucleotide include, but are not limited to, the structures:

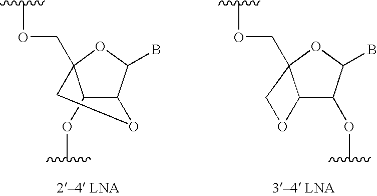

where B is any nucleotide base.

Modifications at the 2′- or 3′-position of ribose include, but are not limited to, hydrogen, hydroxy, methoxy, ethoxy, allyloxy, isopropoxy, butoxy, isobutoxy, methoxyethyl, alkoxy, phenoxy, azido, amino, alkylamino, fluoro, chloro and bromo. Nucleotides include, but are not limited to, the natural D optical isomer, as well as the L optical isomer forms (see, e.g., Garbesi (1993) Nucl. Acids Res. 21:4159-65; Fujimori (1990) J. Amer. Chem. Soc. 112:7435; Urata, (1993) Nucleic Acids Symposium Ser. No. 29:69-70). When the nucleotide base is purine, e.g., A or G, the ribose sugar is attached to the N⁹-position of the nucleotide base. When the nucleotide base is pyrimidine, e.g., C, T or U, the pentose sugar is attached to the N¹-position of the nucleotide base, except for pseudouridines, in which the pentose sugar is attached to the C5 position of the uracil nucleotide base (see, e.g., Kornberg and Baker, (1992) DNA Replication, 2^{nd} Ed., Freeman, San Francisco, Calif.).

One or more of the pentose carbons of a nucleotide may be substituted with a phosphate ester having the formula:

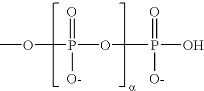

where α is an integer from 0 to 4. In certain embodiments, α is 2 and the phosphate ester is attached to the 3′- or 5′-carbon of the pentose. In certain embodiments, the nucleotides are those in which the nucleotide base is a purine, a 7-deazapurine, a pyrimidine, or an analog thereof. “Nucleotide 5′-triphosphate” refers to a nucleotide with a triphosphate ester group at the 5′ position, and are sometimes denoted as “NTP”, or “dNTP” and “ddNTP” to particularly point out the structural features of the ribose sugar. The triphosphate ester group may include sulfur substitutions for the various oxygens, e.g., α-thio-nucleotide 5′-triphosphates. For a review of nucleotide chemistry, see: Shabarova, Z. and Bogdanov, A. Advanced Organic Chemistry of Nucleic Acids, VCH, New York, 1994.

The term “nucleotide analog”, as used herein, refers to embodiments in which the pentose sugar and/or the nucleotide base and/or one or more of the phosphate esters of a nucleotide may be replaced with its respective analog. In certain embodiments, exemplary pentose sugar analogs are those described above. In certain embodiments, the nucleotide analogs have a nucleotide base analog as described above. In certain embodiments, exemplary phosphate ester analogs include, but are not limited to, alkylphosphonates, methylphosphonates, phosphoramidates, phosphotriesters, phosphorothioates, phosphorodithioates, phosphoroselenoates, phosphorodiselenoates, phosphoroanilothioates, phosphoroanilidates, phosphoroamidates, boronophosphates, etc., and may include associated counterions.

Also included within the definition of “nucleotide analog” are nucleotide analog monomers which can be polymerized into polynucleotide analogs in which the DNA/RNA phosphate ester and/or sugar phosphate ester backbone is replaced with a different type of internucleotide linkage. Exemplary polynucleotide analogs include, but are not limited to, peptide nucleic acids, in which the sugar phosphate backbone of the polynucleotide is replaced by a peptide backbone.

As used herein, the terms “polynucleotide”, “oligonucleotide”, and “nucleic acid” are used interchangeably and mean single-stranded and double-stranded polymers of nucleotide monomers, including 2′-deoxyribonucleotides (DNA) and ribonucleotides (RNA) linked by internucleotide phosphodiester bond linkages, or internucleotide analogs, and associated counter ions, e.g., H⁺, NH₄⁺, trialkylammonium, Mg²⁺, Na⁺and the like. A nucleic acid may be composed entirely of deoxyribonucleotides, entirely of ribonucleotides, or chimeric mixtures thereof. The nucleotide monomer units may comprise any of the nucleotides described herein, including, but not limited to, naturally occurring nucleotides and nucleotide analogs. nucleic acids typically range in size from a few monomeric units, e.g., 5-40 when they are sometimes referred to in the art as oligonucleotides, to several thousands of monomeric nucleotide units. Unless denoted otherwise, whenever a nucleic acid sequence is represented, it will be understood that the nucleotides are in 5′ to 3′ order from left to right and that “A” denotes deoxyadenosine or an analog thereof, “C” denotes deoxycytidine or an analog thereof, “G” denotes deoxyguanosine or an analog thereof, and “T” denotes thymidine or an analog thereof, unless otherwise noted.

Nucleic acids include, but are not limited to, genomic DNA, cDNA, hnRNA, mRNA, rRNA, tRNA, fragmented nucleic acid, nucleic acid obtained from subcellular organelles such as mitochondria or chloroplasts, and nucleic acid obtained from microorganisms or DNA or RNA viruses that may be present on or in a biological sample.

Nucleic acids may be composed of a single type of sugar moiety, e.g., as in the case of RNA and DNA, or mixtures of different sugar moieties, e.g., as in the case of RNA/DNA chimeras. In certain embodiments, nucleic acids are ribopolynucleotides and 2′-deoxyribopolynucleotides according to the structural formulae below:

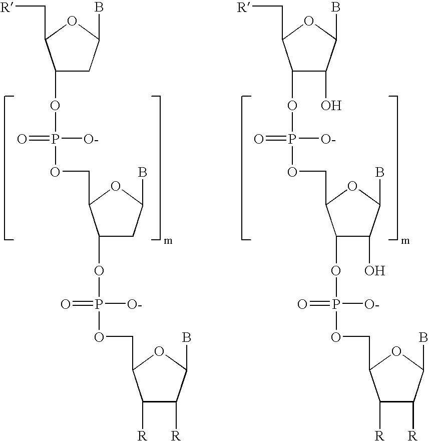

wherein each B is independently the base moiety of a nucleotide, e.g., a purine, a 7-deazapurine, a pyrimidine, or an analog nucleotide; each m defines the length of the respective nucleic acid and can range from zero to thousands, tens of thousands, or even more; each R is independently selected from the group comprising hydrogen, halogen, —R″, —OR″, and —NR″R″, where each R″ is independently (C1-C6) alkyl or (C5 -C14) aryl, or two adjacent Rs are taken together to form a bond such that the ribose sugar is 2′,3′-didehydroribose; and each R′ is independently hydroxyl or

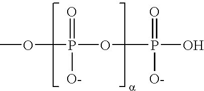

where a is zero, one or two.

In certain embodiments of the ribopolynucleotides and 2′-deoxyribopolynucleotides illustrated above, the nucleotide bases B are covalently attached to the C1′ carbon of the sugar moiety as previously described.

The terms “nucleic acid”, “polynucleotide”, and “oligonucleotide” may also include nucleic acid analogs, polynucleotide analogs, and oligonucleotide analogs. The terms “nucleic acid analog”, “polynucleotide analog” and “oligonucleotide analog” are used interchangeably and, as used herein, refer to a nucleic acid that contains at least one nucleotide analog and/or at least one phosphate ester analog and/or at least one pentose sugar analog. Also included within the definition of nucleic acid analogs are nucleic acids in which the phosphate ester and/or sugar phosphate ester linkages are replaced with other types of linkages, such as N-(2-aminoethyl)-glycine amides and other amides (see, e.g., Nielsen et al., 1991, Science 254: 1497-1500; WO 92/20702; U.S. Pat. No. 5,719,262; U.S. Pat. No. 5,698,685;); morpholinos (see, e.g., U.S. Pat. No. 5,698,685; U.S. Pat. No. 5,378,841; U.S. Pat. No. 5,185,144); carbamates (see, e.g., Stirchak & Summerton, 1987, J. Org. Chem. 52: 4202); methylene(methylimino) (see, e.g., Vasseur et al., 1992, J. Am. Chem. Soc. 114: 4006); 3′-thioformacetals (see, e.g., Jones et al., 1993, J. Org. Chem. 58: 2983); sulfamates (see, e.g., U.S. Pat. No. 5,470,967); 2-aminoethylglycine, commonly referred to as PNA (see, e.g., Buchardt, WO 92/20702; Nielsen (1991) Science 254:1497-1500); and others (see, e.g., U.S. Pat. No. 5,817,781; Frier & Altman, 1997, Nucl. Acids Res. 25:4429 and the references cited therein). Phosphate ester analogs include, but are not limited to, (i) C₁-C₄ alkylphosphonate, e.g., methylphosphonate; (ii) phosphoramidate; (iii) C₁-C₆ alkyl-phosphotriester; (iv) phosphorothioate; and (v) phosphorodithioate.

An “enzymatically active mutant or variant thereof,” when used in reference to an enzyme such as a polymerase or a ligase, means a protein with appropriate enzymatic activity. Thus, for example, but without limitation, an enzymatically active mutant or variant of a DNA polymerase is a protein that is able to catalyze the stepwise addition of appropriate deoxynucleoside triphosphates into a nascent DNA strand in a template-dependent manner. An enzymatically active mutant or variant differs from the “generally-accepted” or consensus sequence for that enzyme by at least one amino acid, including, but not limited to, substitutions of one or more amino acids, addition of one or more amino acids, deletion of one or more amino acids, and alterations to the amino acids themselves. With the change, however, at least some catalytic activity is retained. In certain embodiments, the changes involve conservative amino acid substitutions. Conservative amino acid substitution may involve replacing one amino acid with another that has, e.g., similar hydrophobicity, hydrophilicity, charge, or aromaticity. In certain embodiments, conservative amino acid substitutions may be made on the basis of similar hydropathic indices. A hydropathic index takes into account the hydrophobicity and charge characteristics of an amino acid, and in certain embodiments, may be used as a guide for selecting conservative amino acid substitutions. The hydropathic index is discussed, e.g., in Kyte et al., J. Mol. Biol., 157:105-131 (1982). It is understood in the art that conservative amino acid substitutions may be made on the basis of any of the aforementioned characteristics.

Alterations to the amino acids may include, but are not limited to, glycosylation, methylation, phosphorylation, biotinylation, and any covalent and noncovalent additions to a protein that do not result in a change in amino acid sequence. “Amino acid” as used herein refers to any amino acid, natural or non-natural, that may be incorporated, either enzymatically or synthetically, into a polypeptide or protein.

Fragments, for example, but without limitation, proteolytic cleavage products, are also encompassed by this term, provided that at least some enzyme catalytic activity is retained.

The skilled artisan will readily be able to measure catalytic activity using an appropriate well-known assay. Thus, an appropriate assay for polymerase catalytic activity might include, for example, measuring the ability of a variant to incorporate, under appropriate conditions, rNTPs or dNTPs into a nascent polynucleotide strand in a template-dependent manner. Likewise, an appropriate assay for ligase catalytic activity might include, for example, the ability to ligate adjacently hybridized oligonucleotides comprising appropriate reactive groups. Protocols for such assays may be found, among other places, in Sambrook et al., Molecular Cloning, A Laboratory Manual, Cold Spring Harbor Press (1989) (hereinafter “Sambrook et al.”), Sambrook and Russell, Molecular Cloning, Third Edition, Cold Spring Harbor Press (2000) (hereinafter “Sambrook and Russell”), Ausbel et al., Current Protocols in Molecular Biology (1993) including supplements through April 2001, John Wiley & Sons (hereinafter “Ausbel et al.”).

As used herein, the term “methylation state” refers to the presence or absence of a methyl group on a particular cytosine.

A “target” or “target nucleic acid sequence” according to the present invention comprises a specific nucleic acid sequence that can be distinguished by a probe. In certain embodiments, a target nucleic acid sequence is naturally occurring. In certain embodiments, a target nucleic acid sequence comprises synthetic molecules.

The term “target cytosine” means a cytosine of a target nucleic acid sequence, the methylation state of which is sought to be determined.

A sample may contain a mixture of target nucleic acid sequences, some of which are methylated at a particular target cytosine and some of which are not methylated at that target cytosine. As used herein, the term “degree of methylation” refers to the relative number of target nucleic acid sequences within a sample that are methylated at a target cytosine, compared to those that are not methylated at that target cytosine.

The term “combined methylation” refers to the degree of methylation of two or more different target cytosines in a sample. In certain embodiments, the term “overall degree of methylation” includes the degree of methylation of all of the cytosines in a sample.

The terms “tag” and “tag complement,” as used herein, refer to single-stranded nucleic acids that complement another single-stranded nucleic acid. In certain embodiments, the term “tag complement” refers to a nucleic acid that is complementary to the nucleic acid designated as the “tag.”

“Probes”, according to the present invention, comprise oligonucleotides that comprise a specific portion that is designed to hybridize in a sequence-specific manner with a complementary region on a specific nucleic acid sequence, e.g., a target nucleic acid sequence. In certain embodiments, the specific portion of the probe may be specific for a particular sequence, or alternatively, may be degenerate, e.g., specific for a set of sequences.

A “ligation probe set” according to the present invention is a group of two or more probes designed to detect at least one target. As a non-limiting example, a ligation probe set may comprise two nucleic acid probes designed to hybridize to a target such that, when the two probes are hybridized to the target adjacent to one another, they are suitable for ligation together.

When used in the context of the present invention, “suitable for ligation” refers to at least one first target-specific probe and at least one second target-specific probe, each comprising an appropriately reactive group. Exemplary reactive groups include, but are not limited to, a free hydroxyl group on the 3′ end of the first probe and a free phosphate group on the 5′ end of the second probe. Exemplary pairs of reactive groups include, but are not limited to: phosphorothioate and tosylate or iodide; esters and hydrazide; RC(O)S⁻, haloalkyl, or RCH₂S and α-haloacyl; thiophosphoryl and bromoacetoamido groups. Exemplary reactive groups include, but are not limited to, S-pivaloyloxymethyl-4-thiothymidine. Additionally, in certain embodiments, first and second target-specific probes are hybridized to the target sequence such that the 3′ end of the first target-specific probe and the 5′ end of the second target-specific probe are immediately adjacent to allow ligation.

The term “signal moiety” as used herein refers to any tag, label, or identifiable moiety.

“Detectably different signal” means that detectable signals from different labels are distinguishable from one another by at least one detection method.

The term “detectable signal value” refers to a value of the signal that is detected from a label. In certain embodiments, the detectable signal value is the amount or intensity of signal that is detected from a label. Thus, if there is no detectable signal from a label, its detectable signal value is zero (0). In certain embodiments, the detectable signal value is a characteristic of the signal other than the amount or intensity of the signal, such as the spectra, wavelength, color, or lifetime of the signal.

“Detectably different signal value” means that one or more detectable signal values are distinguishable from one another by at least one detection method.

The term “labeled probe” refers to a probe that provides a detectably different signal value depending upon whether a given nucleic acid sequence is present or absent. In certain embodiments, a labeled probe provides a detectably different signal value when the intact labeled probe is hybridized to a given nucleic acid sequence than when the intact labeled probe is not hybridized to a given nucleic acid sequence. Thus, if a given nucleic acid sequence is present, the labeled probe provides a detectably different signal value than when the given nucleic acid sequence is absent. In certain embodiments, a labeled probe provides a detectably different signal value when the probe is intact than when the probe is not intact. In certain such embodiments, a labeled probe remains intact unless a given nucleic acid sequence is present. In certain such embodiments, if a given nucleic acid sequence is present, the labeled probe is cleaved, which results in a detectably different signal value than when the probe is intact.

The term “double-stranded-dependent label” refers to a label that provides a detectably different signal value when it is exposed to double-stranded nucleic acid than when it is not exposed to double-stranded nucleic acid.

The term “threshold difference between detectable signal values” refers to a set difference between a first detectable signal value and a second detectable signal value that results when the target nucleic acid sequence that is being sought is present in a sample, but that does not result when the target nucleic acid sequence is absent. The first detectable signal value of a double-stranded-dependent label is the detectable signal value from the label when it is not exposed to double-stranded nucleic acid. The second detectable signal value is detected during and/or after an amplification reaction using a composition that comprises the double-stranded-dependent label.

The term “quantitating,” when used in reference to an amplification product, refers to determining the quantity or amount of a particular sequence that is representative of a target nucleic acid sequence in the sample. For example, but without limitation, one may measure the intensity of the signal from a label. The intensity or quantity of the signal is typically related to the amount of amplification product. The amount of amplification product generated correlates with the amount of target nucleic acid sequence present prior to ligation and amplification, and thus, in certain embodiments, may indicate the level of expression for a particular gene.

The terms “annealing” and “hybridization” are used interchangeably and mean the base-pairing interaction of one nucleic acid with another nucleic acid that results in formation of a duplex, triplex, or other higher-ordered structure. In certain embodiments, the primary interaction is base specific, e.g., A/T and G/C, by Watson/Crick and Hoogsteen-type hydrogen bonding. In certain embodiments, base-stacking and hydrophobic interactions may also contribute to duplex stability.

The term “amplification product” as used herein refers to the product of an amplification (PCR) reaction including, but not limited to, primer extension, the polymerase chain reaction, RNA transcription, and the like. Thus, exemplary amplification products may comprise at least one of primer extension products, PCR amplicons, RNA transcription products, and the like.

“Primers” according to the present invention refer to oligonucleotides that are designed to hybridize with the primer-specific portion of probes, ligation products, or amplification products in a sequence-specific manner, and serve as primers for amplification reactions.

A “universal primer” is capable of hybridizing to the primer-specific portion of more than one species of probe, ligation product, or amplification product, as appropriate. A “universal primer set” comprises a first primer and a second primer that hybridize with a plurality of species of probes, ligation products, or amplification products, as appropriate.

A “ligation agent” according to the present invention may comprise any number of enzymatic or chemical (i.e., non-enzymatic) agents that can effect ligation of nucleic acids to one another.

In this application, a statement that one sequence is the same as or is complementary to another sequence encompasses situations where both of the sequences are completely the same or complementary to one another, and situations where only a portion of one of the sequences is the same as, or is complementary to, a portion or the entire other sequence. Here, the term “sequence” encompasses, but is not limited to, nucleic acid sequences, polynucleotides, oligonucleotides, probes, primers, primer-specific portions, target-specific portions, addressable portions, and oligonucleotide link elements.

In this application, a statement that one sequence is complementary to another sequence encompasses situations in which the two sequences have mismatches. Here, the term “sequence” encompasses, but is not limited to, nucleic acid sequences, polynucleotides, oligonucleotides, probes, primers, primer-specific portions, target-specific portions, addressable portions, and oligonucleotide link elements. Despite the mismatches, the two sequences should selectively hybridize to one another under appropriate conditions.

The term “selectively hybridize” means that, for particular identical sequences, a substantial portion of the particular identical sequences hybridize to a given desired sequence or sequences, and a substantial portion of the particular identical sequences do not hybridize to other undesired sequences. A “substantial portion of the particular identical sequences” in each instance refers to a portion of the total number of the particular identical sequences, and it does not refer to a portion of an individual particular identical sequence. In certain embodiments, “a substantial portion of the particular identical sequences” means at least 90% of the particular identical sequences. In certain embodiments, “a substantial portion of the particular identical sequences” means at least 95% of the particular identical sequences.

In certain embodiments, the number of mismatches that may be present may vary in view of the complexity of the composition. Thus, in certain embodiments, fewer mismatches may be tolerated in a composition comprising DNA from an entire genome than a composition in which fewer DNA sequences are present. For example, in certain embodiments, with a given number of mismatches, a probe may more likely hybridize to undesired sequences in a composition with the entire genomic DNA than in a composition with fewer DNA sequences, when the same hybridization conditions are employed for both compositions. Thus, that given number of mismatches may be appropriate for the composition with fewer DNA sequences, but fewer mismatches may be more optimal for the composition with the entire genomic DNA.

In certain embodiments, sequences are complementary if they have no more than 20% mismatched nucleotides. In certain embodiments, sequences are complementary if they have no more than 15% mismatched nucleotides. In certain embodiments, sequences are complementary if they have no more than 10% mismatched nucleotides. In certain embodiments, sequences are complementary if they have no more than 5% mismatched nucleotides.

In this application, a statement that one sequence hybridizes or binds to another sequence encompasses situations where the entirety of both of the sequences hybridize or bind to one another, and situations where only a portion of one or both of the sequences hybridizes or binds to the entire other sequence or to a portion of the other sequence. Here, the term “sequence” encompasses, but is not limited to, nucleic acid sequences, polynucleotides, oligonucleotides, probes, primers, primer-specific portions, target-specific portions, addressable portions, and oligonucleotide link elements.

In certain embodiments, the term “to a measurably lesser extent” encompasses situations in which the event in question is reduced at least 10 fold. In certain embodiments, the term “to a measurably lesser extent” encompasses situations in which the event in question is reduced at least 100 fold.

In certain embodiments, a statement that a component may be, is, or has been “substantially removed” means that at least 90% of the component may be, is, or has been removed. In certain embodiments, a statement that a component may be, is, or has been “substantially removed” means that at least 95% of the component may be, is, or has been removed.

“Mobility modifiers” mean any moieties that alter the migration of a polynucleotide in a mobility-dependent analysis technique, such as electrophoresis.

“Mobility-dependent analysis technique” refers to any analysis based on different rates of migration between different analytes. Exemplary mobility-dependent analyses include, but are not limited to, electrophoresis, mass spectroscopy, chromatography, sedimentation, gradient centrifugation, field-flow fractionation, and multi-stage extraction techniques.

The term “capture moiety” means any molecule that can be used to at least partially isolate a nucleic acid. In certain embodiments, the term “capture moiety” includes affinity sets.

As used herein, an “affinity set” is a set of molecules that specifically bind to one another. Affinity sets include, but are not limited to, biotin and avidin, biotin and streptavidin, receptor and ligand, antibody and ligand, antibody and antigen, and a polynucleotide sequence and its complement. One or more members of an affinity set may be coupled to a solid support. Exemplary solid supports include, but are not limited to, agarose, sepharose, magnetic beads, polystyrene, polyacrylamide, glass, membranes, silica, semiconductor materials, silicon, and organic polymers.

### B. Certain Exemplary Components

In certain embodiments, target nucleic acid sequences include, but are not limited to DNA. Exemplary DNA target sequences include, but are not limited to, genomic DNA, plasmid DNA, phage DNA, nucleolar DNA, mitochondrial DNA, chloroplast DNA, synthetic DNA, and DNA amplification products.

In certain embodiments, target nucleic acid sequences include, but are not limited to, cDNA, yeast artificial chromosomes (YAC's), bacterial artificial chromosomes (BAC's), other extrachromosomal DNA, and nucleic acid analogs. Exemplary nucleic acid analogs include, but are not limited to, LNAs, PNAs, PPG's, and other nucleic acid analogs. PPG is pyrrazolopyrimidine dG, which is discussed, e.g., in Sedelnikova et al., Antisense Nucleic Acid Drug Dev 2000, 10(6):443-452 (December 2000).

In certain embodiments, target nucleic acid sequences include, but are not limited to RNA. Exemplary RNA target sequences include, but are not limited to, mRNA, rRNA, tRNA, viral RNA, and variants of RNA, such as splicing variants.

A variety of methods are available for obtaining a target nucleic acid sequence for use with the compositions and methods of the present invention. When the nucleic acid target is obtained through isolation from a biological matrix, certain isolation techniques include, but are not limited to, (1) organic extraction followed by ethanol precipitation, e.g., using a phenol/chloroform organic reagent (e.g., Ausubel et al., eds., Current Protocols in Molecular Biology Volume 1, Chapter 2, Section I, John Wiley & Sons, New York (1993)), in certain embodiments, using an automated DNA extractor, e.g., the Model 341 DNA Extractor available from Applied Biosystems (Foster City, Calif.); (2) stationary phase adsorption methods (e.g., Boom et al., U.S. Pat. No. 5,234,809; Walsh et al., Biotechniques 10(4): 506-513 (1991)); and (3) salt-induced DNA precipitation methods (e.g., Miller et al., Nucleic Acids Research,16(3): 9-10 (1988)), such precipitation methods being typically referred to as “salting-out” methods. In certain embodiments, the above isolation methods may be preceded by an enzyme digestion step to help eliminate unwanted protein from the sample, e.g., digestion with proteinase K, or other like proteases. See, e.g., U.S. patent application Ser. No. 09/724,613.

In certain embodiments, a target nucleic acid sequence may be derived from any living, or once living, organism, including but not limited to prokaryote, eukaryote, plant, animal, and virus. In certain embodiments, the target nucleic acid sequence may originate from a nucleus of a cell, e.g., genomic DNA, or may be extranuclear nucleic acid, e.g., plasmid, mitrochondrial nucleic acid, various RNAs, and the like. In certain embodiments, if the sequence from the organism is RNA, it may be reverse-transcribed into a cDNA target nucleic acid sequence. Furthermore, in certain embodiments, the target nucleic acid sequence may be present in a double stranded or single stranded form.

Exemplary target nucleic acid sequences include, but are not limited to, amplification products, ligation products, transcription products, reverse transcription products, primer extension products, methylated DNA, and cleavage products. Exemplary amplification products include, but are not limited to, PCR and isothermal products.

In certain embodiments, nucleic acids in a sample may be subjected to a cleavage procedure. In certain embodiments, such cleavage products may be targets.

Different target nucleic acid sequences may be different portions of a single contiguous nucleic acid or may be on different nucleic acids. Different portions of a single contiguous nucleic acid may or may not overlap.

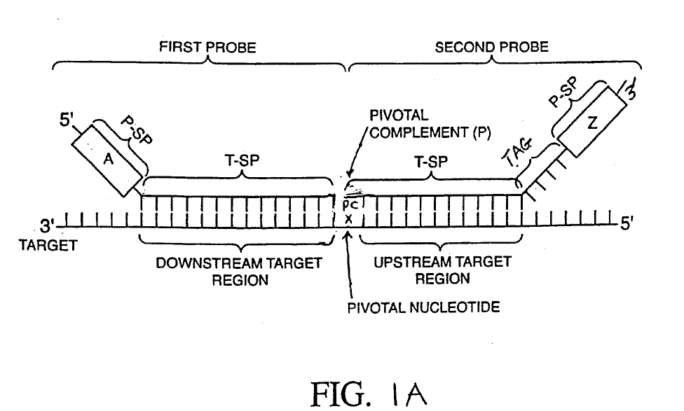

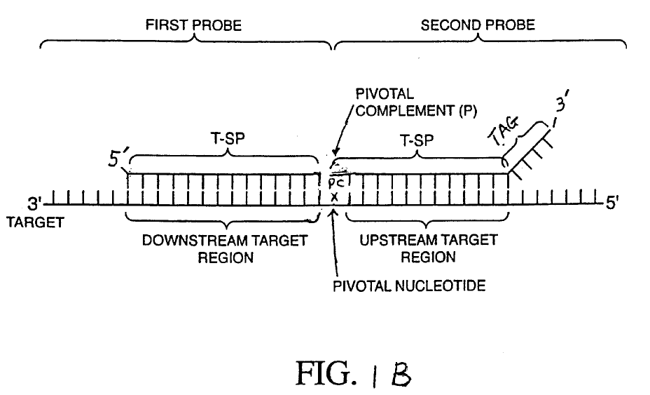

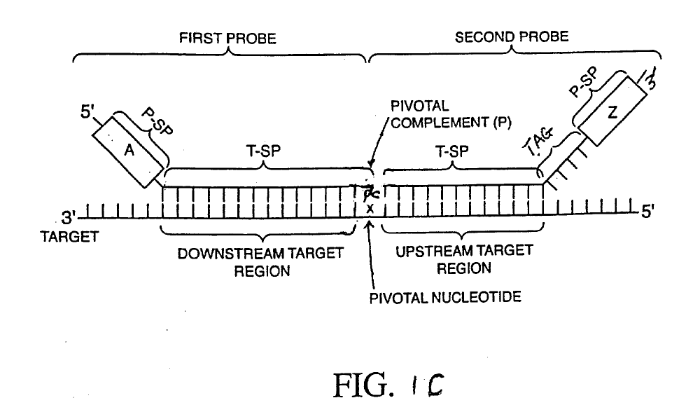

In certain embodiments, a target nucleic acid sequence comprises an upstream or 5′ region, a downstream or 3′ region, and a “pivotal nucleotide” located in the upstream region or the downstream region (see, e.g., FIGS. 1(A)-(D). In certain embodiments, the pivotal nucleotide may be the nucleotide being detected by the probe set to determine the methylation state of a target cytosine. In certain embodiments, more than one pivotal nucleotide is present. In certain embodiments, one or more pivotal nucleotides is located in the upstream region, and one or more pivotal nucleotide is located in the downstream region. In certain embodiments, more than one pivotal nucleotides is located in the upstream region or the downstream region.

The person of ordinary skill will appreciate that while a target nucleic acid sequence is typically described as a single-stranded molecule, the opposing strand of a double-stranded molecule comprises a complementary sequence that may also be used as a target sequence.

A ligation probe set, according to certain embodiments, comprises two or more probes that comprise a target-specific portion that is designed to hybridize in a sequence-specific manner with a complementary region on a specific target nucleic acid sequence (see, e.g., first and second probes in FIGS. 1(A)-(D)). In certain embodiments, a probe of a ligation probe set may further comprise a primer-specific portion, a tag sequence, all or part of a promoter or its complement, or a combination of these additional components. In certain embodiments, any of the probe's components may overlap any other probe component(s). For example, but without limitation, the target-specific portion may overlap the primer-specific portion, the promoter or its complement, or both. Also, without limitation, the tag sequence may overlap with the target-specific portion or the primer specific-portion, or both.

In certain embodiments, at least one probe of a ligation probe set comprises the tag sequence located between the target-specific portion and the primer-specific portion (see, e.g., second probe in FIGS. 1(A) and 1(C)). In certain embodiments, the probe's tag sequence may comprise a sequence that is the same as, or complementary to, a portion of a capture oligonucleotide sequence located on an addressable support or a bridging oligonucleotide. In certain embodiments, the probe's tag sequence may comprise a mobility modifier that allows detection of the ligation or amplification products based on their location at a particular mobility address due to a mobility detection process, such as, but without limitation, electrophoresis. In certain embodiments, one employs a mobility-modifier comprising (1) a tag complement or tag sequence for selectively binding to the tag sequence or tag complement of a ligation product and/or an amplification product, and (2) a tail for effecting a particular mobility in a mobility-dependent analysis technique, e.g., electrophoresis, see, e.g., U.S. patent application No. 09/522,640, filed Mar. 15, 1999. In certain embodiments, the probe's tag sequence is not complementary with target nucleic acid sequences, primer sequences, or probe sequences.

The sequence-specific portions of probes are of sufficient length to permit specific annealing to complementary sequences in primers, mobility modifier cassettes, and targets as appropriate. In certain embodiments, the length of the tag sequences are 6 to 35 nucleotides. In certain embodiments, the length of the target-specific portions are 6 to 35 nucleotides. Detailed descriptions of probe design that provide for sequence-specific annealing can be found, among other places, in Diffenbach and Dveksler, PCR Primer, A Laboratory Manual, Cold Spring Harbor Press, 1995, and Kwok et al., Nucl. Acid Res. 18:999-1005 (1990).

A ligation probe set according to certain embodiments comprises at least one first probe and at least one second probe that adjacently hybridize to the same target nucleic acid sequence. According to certain embodiments, a ligation probe set is designed so that the target-specific portion of the first probe will hybridize with the downstream target region (see, e.g., FIGS. 1(A)-(D)) and the target-specific portion of the second probe will hybridize with the upstream target region (see, e.g., FIGS. 1(A)-(D)). The sequence-specific portions of the probes are of sufficient length to permit specific annealing with complementary sequences in targets and primers, as appropriate. In certain embodiments, one of the at least one first probe and the at least one second probe in a probe set further comprises a tag sequence.

Under appropriate conditions, adjacently hybridized probes may be ligated together to form a ligation product, provided that they comprise appropriate reactive groups, for example, without limitation, a free 3′-hydroxyl or 5′-phosphate group.

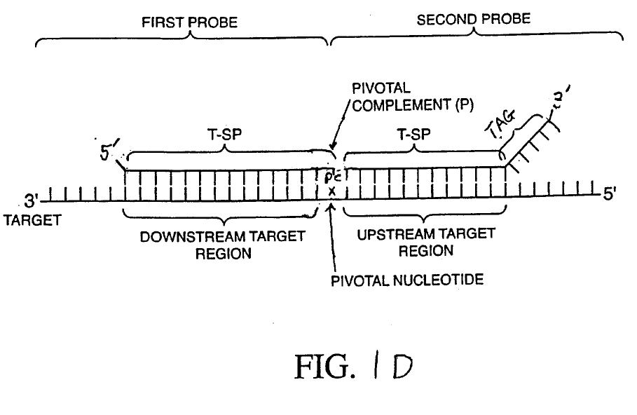

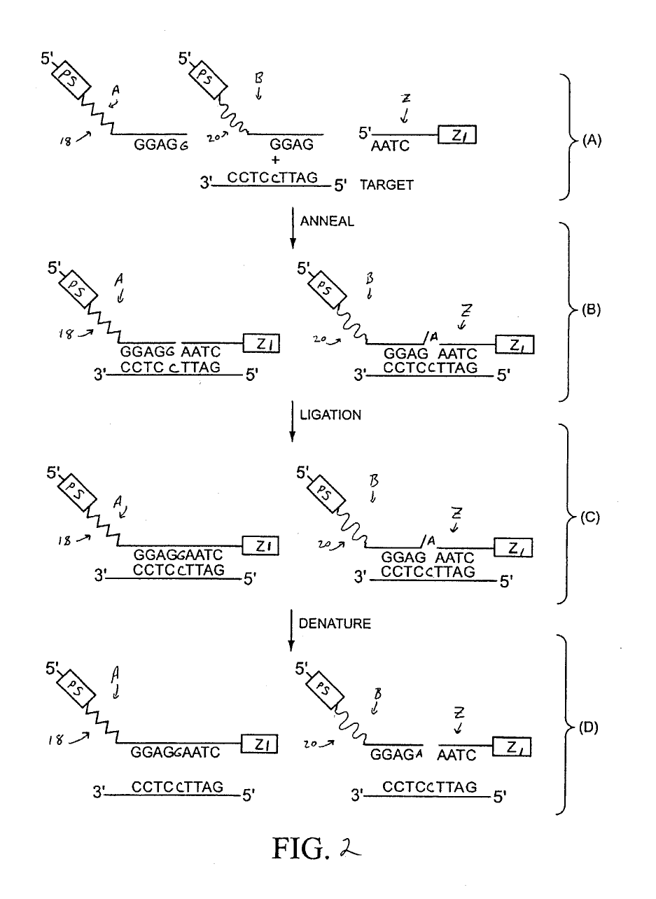

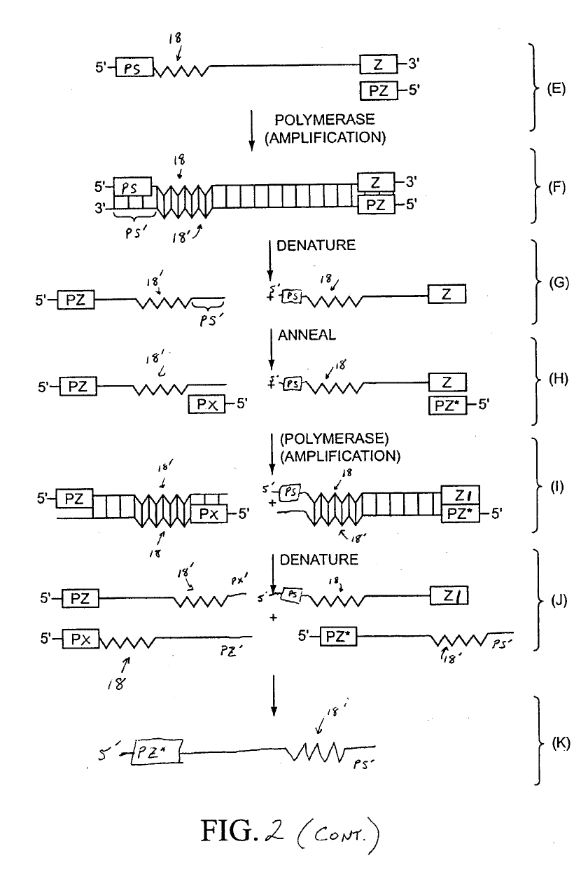

According to certain embodiments, some ligation probe sets may comprise more than one first probe or more than one second probe to allow sequence discrimination between target sequences that differ by one or more nucleotides (see, e.g., FIGS. 2-5).

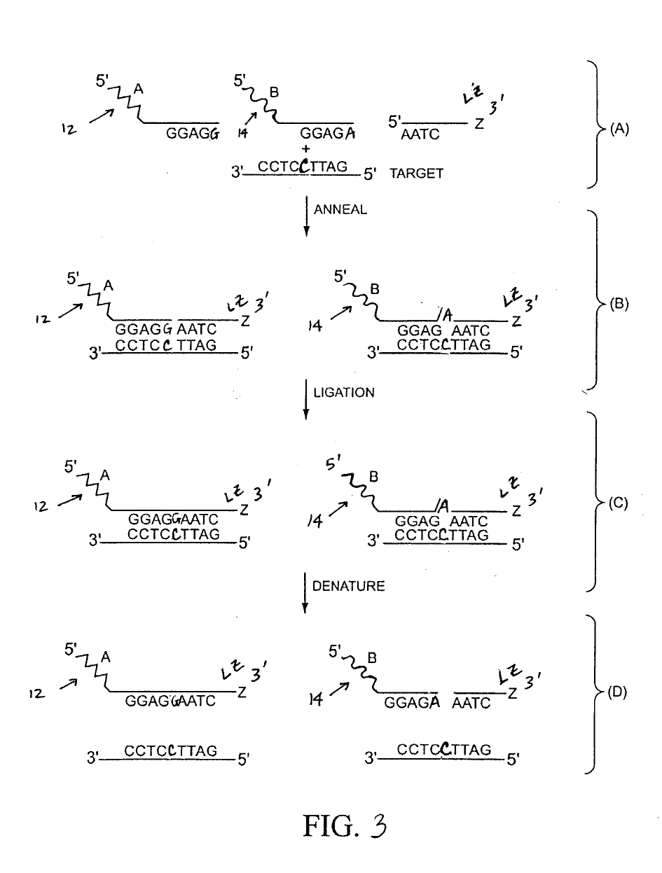

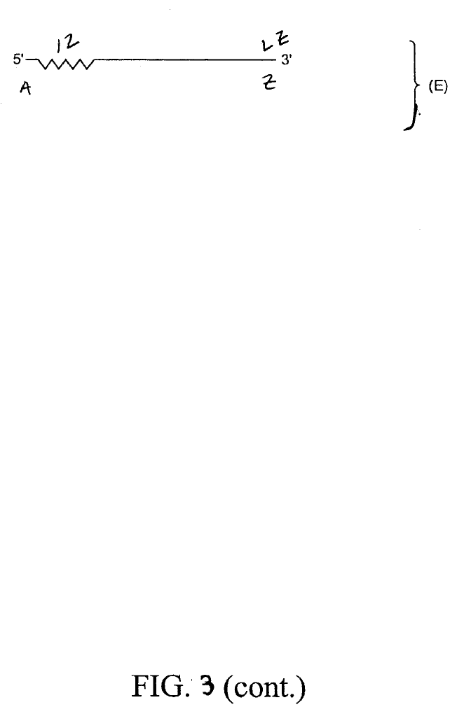

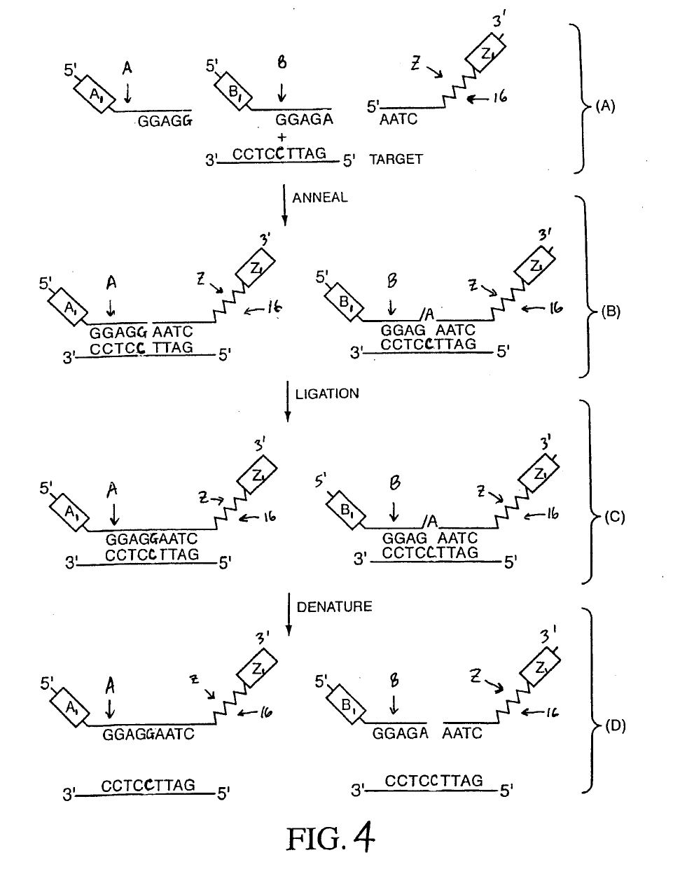

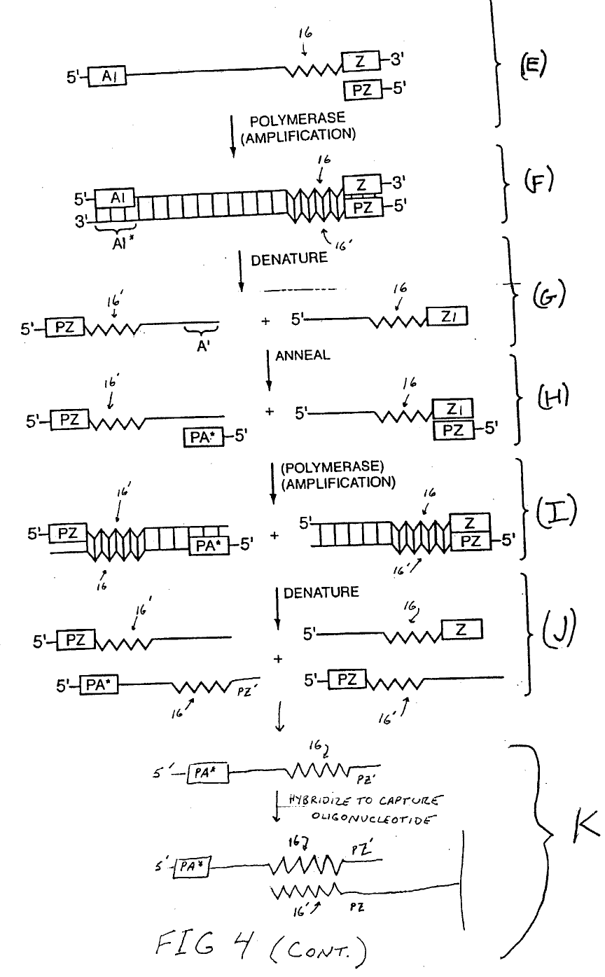

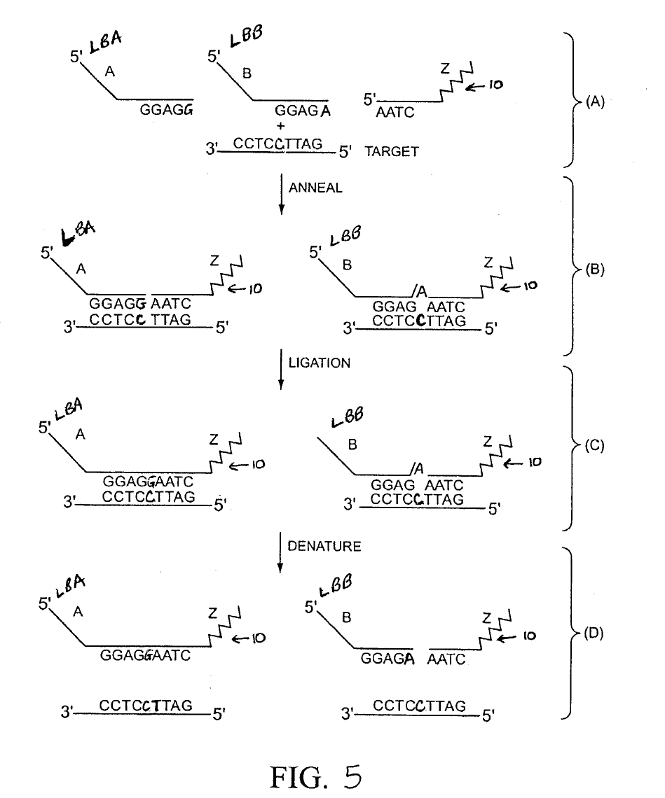

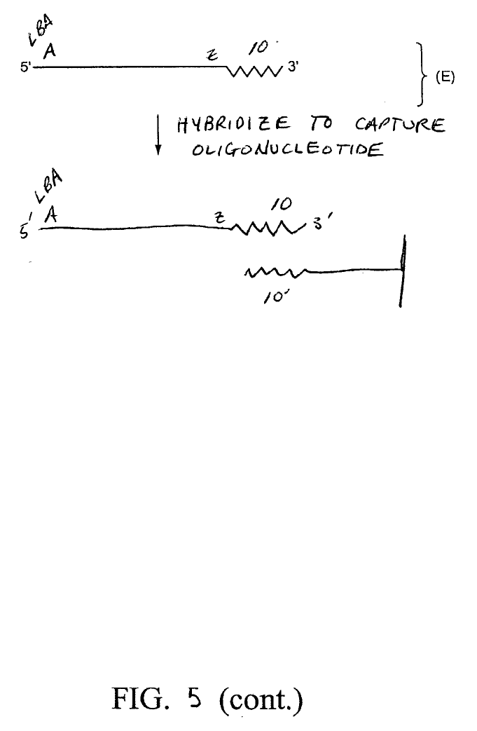

In certain embodiments, a nucleotide base complementary to the pivotal nucleotide, the “pivotal complement” or “pivotal complement nucleotide,” is present on the proximal end of the second probe of the target-specific probe set (see, e.g., 5′ end (PC) of the second probe in FIGS. 1(A) and (B)). In certain embodiments, the second probe further comprises a tag sequence (see, e.g., FIGS. 1(A) and (B)). In certain embodiments, the first probe may comprise a pivotal complement and the second probe may comprise a tag sequence (see, e.g., FIGS. 1(C) and (D) and FIGS. 4 and 5). In certain embodiments, the first probe may comprise a pivotal complement and a tag sequence (see, e.g., FIGS. 2 and 3). In certain embodiments, the second probe may comprise a pivotal complement and the first probe may comprise a tag sequence.

The skilled artisan will appreciate that the pivotal nucleotide(s) may be located anywhere in the target sequence and that likewise, the pivotal complement may be located anywhere within the target-specific portion of the probe(s). For example, according to various embodiments, the pivotal complement may be located at the 3′ end of a probe, at the 5′ end of a probe, or anywhere between the 3′ end and the 5′ end of a probe.

In certain embodiments, when the first and second probes of the ligation probe set are hybridized to the appropriate upstream and downstream target regions, and when the pivotal complement is at the 5′ end of one probe or the 3′ end of the other probe, and the pivotal complement is base-paired with the pivotal nucleotide on the target sequence, the hybridized first and second probes may be ligated together to form a ligation product (see, e.g., FIGS. 2(B)-(C), 3(B)-(C), 4(B)-(C), and 5(B)-(C)). In the example shown in FIGS. 2(B)-(C) 3(B)-(C), 4(B)-(C), and 5(B)-(C), a mismatched base at the pivotal nucleotide, however, interferes with ligation, even if both probes are otherwise fully hybridized to their respective target regions.

In certain embodiments, other mechanisms may be employed to avoid ligation of probes that do not include the correct complementary nucleotide at the pivotal complement. For example, in certain embodiments, conditions may be employed such that a probe of a ligation probe set will hybridize to the target sequence to a measurably lesser extent if there is a mismatch at the pivotal nucleotide. Thus, in such embodiments, such non-hybridized probes will not be ligated to the other probe in the probe set.

In certain embodiments, the first probes and second probes in a ligation probe set are designed with similar melting temperatures (Tₘ). Where a probe includes a pivotal complement, in certain embodiments, the Tₘ for the probe(s) comprising the pivotal complement(s) of the target pivotal nucleotide sought will be approximately 4-15° C. lower than the other probe(s) that do not contain the pivotal complement in the probe set. In certain such embodiments, the probe comprising the pivotal complement(s) will also be designed with a Tₘ near the ligation temperature. Thus, a probe with a mismatched nucleotide will more readily dissociate from the target at the ligation temperature. The ligation temperature, therefore, in certain embodiments provides another way to discriminate between, for example, target cytosine methylation states in the target.

Further, in certain embodiments, ligation probe sets do not comprise a pivotal complement at the terminus of the first or the second probe (e.g., at the 3′ end or the 5′ end of the first or second probe). Rather, the pivotal complement is located somewhere between the 5′ end and the 3′ end of the first or second probe. In certain such embodiments, probes with target-specific portions that are fully complementary with their respective target regions will hybridize under high stringency conditions. Probes with one or more mismatched bases in the target-specific portion, by contrast, will hybridize to their respective target region to a measurably lesser extent. Both the first probe and the second probe must be hybridized to the target for a ligation product to be generated.

In certain embodiments, highly related sequences that differ by as little as a single nucleotide can be distinguished. In certain embodiments, one can combine a ligation probe set comprising two first probes, differing in their tag sequences and their pivotal complement (see, e.g., probes A and B in FIGS. 2(A), 3(A), 4(A), and 5(A)), one second probe (see, e.g., probe Z in FIGS. 2(A), 3(A), 4(A), and 5(A)), and the sample containing the target. All three probes will hybridize with the target sequence under appropriate conditions (see, e.g., FIGS. 2(B), 3(B), 4(B), and 5(B). Only the first probe with the hybridized pivotal complement, however, will be ligated with the hybridized second probe (see, e.g., FIGS. 2(C), 3(C), 4(C), and 5(C)). Thus, if only one allele is present in the sample, only one ligation product for that target will be generated (see, e.g., ligation product A-Z in FIGS. 2(D), 3(D), 4(D), and 5(D)). Both ligation products would be formed in a sample from an individual with different methylation states at a given target cytosine. In certain embodiments, ligation of probes with a pivotal complement that is not complementary to the pivotal nucleotide may occur, but such ligation occurs to a measurably lesser extent than ligation of probes with a pivotal complement that is complementary to the pivotal nucleotide.

In certain embodiments, one of the first probe or the second probe may contain a pivotal complement and the other of the first probe or the second probe may contain a tag sequence. See, e.g., FIGS. 4 and 5.

In certain embodiments, one of the first or second probes of a ligation probe set may include a tag sequence and the first and second probes may not include primer-specific portions. In certain such embodiments, at least one first probe of a probe set comprises a pivotal complement and a tag sequence and at least one second probe of a probe set comprises a label (see, e.g., FIG. 3). In other such embodiments, at least one first probe of a probe set comprises a pivotal complement and a label and at least one second probe of a probe set comprises a tag sequence (see, e.g., FIG. 5).

In certain embodiments, conditions may be employed such that probes of a ligation probe set will hybridize to the target sequence to a measurably lesser extent if the target cytosine is not in the appropriate methylation state. In certain embodiments, conditions may be employed such that probes of a ligation probe set will not hybridize to the target sequence if the target cytosine is not in the appropriate methylation state.

In certain embodiments, conditions may be employed such that probes of a ligation probe set will ligate together to a measurably lesser extent if the target cytosine is not in the appropriate methylation state, even if the probes of the probe set are hybridized to the target sequence. In certain embodiments, conditions may be employed such that probes of a ligation probe set will not ligate together if the target cytosine is not in the appropriate methylation state, even if the probes of the probe set are hybridized to the target sequence.

In certain embodiments, conditions may be employed such that probes of a ligation probe set will hybridize to the target sequence to a measurably lesser extent if the target cytosine is methylated. In certain embodiments, conditions may be employed such that probes of a ligation probe set will not hybridize to the target sequence if the target cytosine is methylated.

In certain embodiments, conditions may be employed such that probes of a ligation probe set will ligate together to a measurably lesser extent if the target cytosine methylated, even if the probes of the probe set are hybridized to the target sequence. In certain embodiments, conditions may be employed such that probes of a ligation probe set will not ligate together if the target cytosine is methylated, even if the probes of the probe set are hybridized to the target sequence.

In certain embodiments, conditions may be employed such that probes of a ligation probe set will hybridize to the target sequence to a measurably lesser extent if the target cytosine is unmethylated. In certain embodiments, conditions may be employed such that probes of a ligation probe set will not hybridize to the target sequence if the target cytosine is unmethylated.

In certain embodiments, conditions may be employed such that probes of a ligation probe set will ligate together to a measurably lesser extent if the target cytosine unmethylated, even if the probes of the probe set are hybridized to the target sequence. In certain embodiments, conditions may be employed such that probes of a ligation probe set will not ligate together if the target cytosine is unmethylated, even if the probes of the probe set are hybridized to the target sequence.

In certain embodiments, conditions may be employed such that probes of a ligation probe set will hybridize to the target sequence to a measurably lesser extent if the target cytosine is modified. In certain embodiments, conditions may be employed such that probes of a ligation probe set will not hybridize to the target sequence if the target cytosine modified.

In certain embodiments, conditions may be employed such that probes of a ligation probe set will ligate together to a measurably lesser extent if the target cytosine is modified, even if the probes of the probe set are hybridized to the target sequence. In certain embodiments, conditions may be employed such that probes of a ligation probe set will not ligate together if the target cytosine is modified, even if the probes of the probe set are hybridized to the target sequence.

In certain embodiments, conditions may be employed such that probes of a ligation probe set will hybridize to the target sequence to a measurably lesser extent if the target cytosine is unmodifed. In certain embodiments, conditions may be employed such that probes of a ligation probe set will not hybridize to the target sequence unless the target cytosine is modified.

In certain embodiments, conditions may be employed such that probes of a ligation probe set will ligate together to a measurably lesser extent if the target cytosine is unmodifed, even if the probes of the probe set are hybridized to the target sequence. In certain embodiments, conditions may be employed such that probes of a ligation probe set will not ligate together unless the target cytosine modified, even if the probes of the probe set are hybridized to the target sequence.

In certain embodiments, conditions may be employed such that probes of a ligation probe set will hybridize to the target sequence to a measurably lesser extent if the target cytosine is converted to a converted nucleotide. In certain embodiments, conditions may be employed such that probes of a ligation probe set will not hybridize to the target sequence if the target cytosine is converted to a converted nucleotide.

In certain embodiments, conditions may be employed such that probes of a ligation probe set will ligate together to a measurably lesser extent if the target cytosine is converted to a converted nucleotide, even if the probes of the probe set are hybridized to the target sequence. In certain embodiments, conditions may be employed such that probes of a ligation probe set will not ligate together if the target cytosine is converted to a converted nucleotide, even if the probes of the probe set are hybridized to the target sequence.

In certain embodiments, conditions may be employed such that probes of a ligation probe set will hybridize to the target sequence to a measurably lesser extent if the target cytosine has not been converted to a converted nucleotide. In certain embodiments, conditions may be employed such that probes of a ligation probe set will not hybridize to the target sequence unless the target cytosine is converted to a converted nucleotide.

In certain embodiments, conditions may be employed such that probes of a ligation probe set will ligate together to a measurably lesser extent if the target cytosine has not been converted to a converted nucleotide, even if the probes of the probe set are hybridized to the target sequence. In certain embodiments, conditions may be employed such that probes of a ligation probe set will not ligate together unless the target cytosine is converted to a converted nucleotide, even if the probes of the probe set are hybridized to the target sequence.

A primer set according to certain embodiments comprises at least one primer capable of hybridizing with the primer-specific portion of at least one probe of a ligation probe set. In certain embodiments, a primer set comprises at least one first primer and at least one second primer, wherein the at least one first primer specifically hybridizes with one probe of a ligation probe set (or a complement of such a probe) and the at least one second primer of the primer set specifically hybridizes with a second probe of the same ligation probe set (or a complement of such a probe). In certain embodiments, at least one primer of a primer set further comprises all or part of a promoter sequence or its complement. In certain embodiments, the first and second primers of a primer set have different hybridization temperatures, to permit temperature-based asymmetric PCR reactions.

The skilled artisan will appreciate that while the probes and primers of the invention may be described in the singular form, a plurality of probes or primers may be encompassed by the singular term, as will be apparent from the context. Thus, for example, in certain embodiments, a ligation probe set typically comprises a plurality of first probes and a plurality of second probes.

The criteria for designing sequence-specific primers and probes are well known to persons of ordinary skill in the art. Detailed descriptions of primer design that provide for sequence-specific annealing can be found, among other places, in Diffenbach and Dveksler, PCR Primer, A Laboratory Manual, Cold Spring Harbor Press, 1995, and Kwok et al. (Nucl. Acid Res. 18:999-1005, 1990). The sequence-specific portions of the primers are of sufficient length to permit specific annealing to complementary sequences in ligation products and amplification products, as appropriate.

In embodiments that employ a promoter sequence, the promoter sequence or its complement will be of sufficient length to permit an appropriate polymerase to interact with it. Detailed descriptions of sequences that are sufficiently long for polymerase interaction can be found in, among other places, Sambrook and Russell.

According to certain embodiments, a primer set of the present invention comprises at least one second primer. The second primer in that primer set is designed to hybridize with a 3′ primer-specific portion of a ligation or amplification product in a sequence-specific manner. In certain embodiments, the primer set further comprises at least one first primer. The first primer of a primer set is designed to hybridize with the complement of the 5′ primer-specific portion of that same ligation or amplification product in a sequence-specific manner. In certain embodiments, at least one primer of the primer set comprises a promoter sequence or its complement or a portion of a promoter sequence or its complement. For a discussion of primers comprising promoter sequences, see, e.g., Sambrook and Russell. In certain embodiments, at least one primer of the primer set further comprises a label. In certain embodiments, labels are fluorescent dyes attached to a nucleotide(s) in the primer (see, e.g., L. Kricka, Nonisotopic DNA Probe Techniques, Academic Press, San Diego, Calif. (1992)). In certain embodiments, a label is attached to the primer in such a way as to not to interfere with sequence-specific hybridization or amplification.

A universal primer or primer set may be employed according to certain embodiments. In certain embodiments, a universal primer or a universal primer set hybridizes with all or most of the probes, ligation products, or amplification products in a reaction, as appropriate. When universal primer sets are used in certain amplification reactions, such as, but not limited to, PCR, qualitative or quantitative results may be obtained for a broad range of template concentrations.

Use of labels can be accomplished using any one of a large number of known techniques employing known labels, linkages, linking groups, reagents, reaction conditions, and analysis and purification methods. The term “label” includes, but is not limited to, any moiety that can be attached to a nucleic acid and: (i) provides a detectable signal; (ii) interacts with a second label to modify the detectable signal provided by the second label, e.g., FRET (Fluorescent Resonance Energy Transfer); or (iii) provides a member of a binding complex or affinity set, e.g., affinity, antibody/antigen, ionic complexation, hapten/ligand, e.g., biotin/avidin.

Exemplary, labels include, but are not limited to, light-emitting or light-absorbing compounds which generate or quench a detectable fluorescent, chemiluminescent, or bioluminescent signal (see, e.g., Kricka, L. in Nonisotopic DNA Probe Techniques (1992), Academic Press, San Diego, pp. 3-28). Fluorescent reporter dyes useful as labels include, but are not limited to, fluoresceins (see, e.g., U.S. Pat. Nos. 5,188,934; 6,008,379; and 6,020,481), rhodamines (see, e.g., U.S. Pat. Nos. 5,366,860; 5,847,162; 5,936,087; 6,051,719; and 6,191,278), benzophenoxazines (see, e.g., U.S. Pat. No. 6,140,500), energy-transfer fluorescent dyes, comprising pairs of donors and acceptors (see, e.g., U.S. Pat. Nos. 5,863,727; 5,800,996; and 5,945,526), and cyanines (see, e.g., Kubista, WO 97/45539), as well as any other fluorescent moiety capable of generating a detectable signal. Examples of fluorescein dyes include, but are not limited to, 6-carboxyfluorescein; 2′,4′,1,4,-tetrachlorofluorescein; and 2′,4′,5′,7′,1,4-hexachlorofluorescein.

Exemplary labels also include, but are not limited to, quantum dots. “Quantum dots” refer to semiconductor nanocrystalline compounds capable of emitting a second energy in response to exposure to a first energy. Typically, the energy emitted by a single quantum dot always has the same predictable wavelength. Exemplary semiconductor nanocrystalline compounds include, but are not limited to, crystals of CdSe, CdS, and ZnS. Suitable quantum dots according to certain embodiments are described, e.g., in U.S. Pat. Nos. 5,990,479 and 6,207,392 B1, and in “Quantum-dot-tagged microbeads for multiplexed optical coding of biomolecules,” Han et al., Nature Biotechnology, 19:631-635 (2001).

Exemplary labels also include, but are not limited to, phosphors and luminescent molecules. Exemplary labels also include, but are not limited to, fluorophores, radioisotopes, chromogens, enzymes, antigens, heavy metals, dyes, magnetic probes, phosphorescence groups, chemiluminescent groups, and electrochemical detection moieties. Exemplary fluorophores that are used as reporter groups include, but are not limited to, rhodamine, cyanine 3 (Cy 3), cyanine 5 (Cy 5), fluorescein, Vic™, Liz™, Tamra™, 5-Fam™, 6-Fam™, and Texas Red (Molecular Probes). (Vic™, Liz™, Tamra™, 5-Fam™, and 6-Fam™ are all available from Applied Biosystems, Foster City, Calif.) Exemplary radioisotopes include, but are not limited to, ³²P, ³³P, and ³⁵S. Exemplary labels also include elements of multi-element indirect reporter systems, e.g., biotin/avidin, antibody/antigen, ligand/receptor, enzyme/substrate, and the like, in which the element interacts with other elements of the system in order to effect a detectable signal. One exemplary multi-element reporter system includes a biotin reporter group attached to a primer and an avidin conjugated with a fluorescent label.

The skilled artisan will appreciate that, in certain embodiments, one or more of the primers, probes, deoxyribonucleotide triphosphates, ribonucleotide triphosphates disclosed herein may further comprise one or more labels. Detailed protocols for methods of attaching labels to oligonucleotides and polynucleotides can be found in, among other places, G. T. Hermanson, Bioconjugate Techniques, Academic Press, San Diego, Calif. (1996) and S. L. Beaucage et al., Current Protocols in Nucleic Acid Chemistry, John Wiley & Sons, New York, N.Y. (2000).

Certain non-radioactive labeling methods, techniques, and reagents are reviewed in: Non-Radioactive Labelling, A Practical Introduction, Garman, A. J. (1997) Academic Press, San Diego.

In certain embodiments, a mobility modifier may be employed. In certain embodiments, mobility modifiers may be nucleotides of different lengths effecting different mobilities. In certain embodiments, mobility modifiers may be non-nucleotide polymers, such as a polyethylene oxide (PEO), polyglycolic acid, polyurethane polymers, polypeptides, or oligosaccharides, as non-limiting examples. In certain embodiments, mobility modifiers may work by adding size to a polynucleotide, or by increasing the “drag” of the molecule during migration through a medium without substantially adding to the size. Certain mobility modifiers such as PEO's have been described, e.g., in U.S. Pat. Nos. 5,470,705; 5,580,732; 5,624,800; and 5,989,871.

Linkage of polymers such as PEO's to polynucleotides is well known in the art. Standard DNA chemistry linkages are described, e.g., in Grossman et al., Nucleic Acids Research, 22(21):4527-34 (1994).

Certain embodiments include a ligation agent. For example, ligase is an enzymatic ligation agent that, under appropriate conditions, forms phosphodiester bonds between the 3′-OH and the 5′-phosphate of adjacent nucleotides in DNA or RNA molecules, or hybrids. Exemplary ligases include, but are not limited to, Tth K294R ligase and Tsp AK16D ligase. See, e.g., Luo et al., Nucleic Acids Res., 24(14):3071-3078 (1996); Tong et al., Nucleic Acids Res., 27(3):788-794 (1999); and Published PCT Application No. WO 00/26381. Temperature sensitive ligases, include, but are not limited to, T4 DNA ligase, T7 DNA ligase, and E. coli ligase. In certain embodiments, thermostable ligases include, but are not limited to, Taq ligase, Tth ligase, Tsc ligase, and Pfu ligase. Certain thermostable ligases may be obtained from thermophilic or hyperthermophilic organisms, including but not limited to, prokaryotic, eucaryotic, or archael organisms. Certain RNA ligases may be employed in certain embodiments. In certain embodiments, the ligase is a RNA dependent DNA ligase, which may be employed with RNA template and DNA ligation probes. An exemplary, but nonlimiting example, of a ligase with such RNA dependent DNA ligase activity is T4 DNA ligase. In certain embodiments, the ligation agent is an “activating” or reducing agent.

Chemical ligation agents include, without limitation, activating, condensing, and reducing agents, such as carbodiimide, cyanogen bromide (BrCN), N-cyanoimidazole, imidazole, 1-methylimidazole/carbodiimide/cystamine, dithiothreitol (DTT) and ultraviolet light. Autoligation, i.e., spontaneous ligation in the absence of a ligating agent, is also within the scope of certain embodiments of the invention. Detailed protocols for chemical ligation methods and descriptions of appropriate reactive groups can be found, among other places, in Xu et al., Nucleic Acid Res., 27:875-81 (1999); Gryaznov and Letsinger, Nucleic Acid Res. 21:1403-08 (1993); Gryaznov et al., Nucleic Acid Res. 22:2366-69 (1994); Kanaya and Yanagawa, Biochemistry 25:7423-30 (1986); Luebke and Dervan, Nucleic Acids Res. 20:3005-09 (1992); Sievers and von Kiedrowski, Nature 369:221-24 (1994); Liu and Taylor, Nucleic Acids Res. 26:3300-04 (1999); Wang and Kool, Nucleic Acids Res. 22:2326-33 (1994); Purmal et al., Nucleic Acids Res. 20:3713-19 (1992); Ashley and Kushlan, Biochemistry 30:2927-33 (1991); Chu and Orgel, Nucleic Acids Res. 16:3671-91 (1988); Sokolova et al., FEBS Letters 232:153-55 (1988); Naylor and Gilham, Biochemistry 5:2722-28 (1966); and U.S. Pat. No. 5,476,930.

In certain embodiments, at least one polymerase is included. In certain embodiments, at least one thermostable polymerase is included. Exemplary thermostable polymerases, include, but are not limited to, Taq polymerase, Pfx polymerase, Pfu polymerase, Vent® polymerase, Deep Vent™ polymerase, Pwo polymerase, Tth polymerase, UlTma polymerase and enzymatically active mutants and variants thereof. Descriptions of these polymerases may be found, among other places, at the world wide web URL: the-scientist.com/yr1998/jan/profile 1_{—}980105.html; at the world wide web URL: the-scientist.com/yr2001/jan/profile_{—}010903.html; at the world wide web URL: the-scientist.com/yr2001/sep/profile2_{—}010903.html; at the article The Scientist 12(1):17 (Jan. 5, 1998); and at the article The Scientist 15(17):1 (Sep. 3, 2001).

The skilled artisan will appreciate that the complement of the disclosed probe, target, and primer sequences, or combinations thereof, may be employed in certain embodiments of the invention. For example, without limitation, a genomic DNA sample may comprise both the target sequence and its complement. Thus, in certain embodiments, when a genomic sample is denatured, both the target sequence and its complement are present in the sample as single-stranded sequences. In certain embodiments, ligation probes may be designed to specifically hybridize to an appropriate sequence, either the target sequence or its complement.

### C. Certain Exemplary Component Methods

Ligation according to the present invention comprises any enzymatic or chemical process wherein an internucleotide linkage is formed between the opposing ends of nucleic acid sequences that are adjacently hybridized to a template. Additionally, the opposing ends of the annealed nucleic acid sequences should be suitable for ligation (suitability for ligation is a function of the ligation method employed). The internucleotide linkage may include, but is not limited to, phosphodiester bond formation. Such bond formation may include, without limitation, those created enzymatically by a DNA or RNA ligase, such as bacteriophage T4 DNA ligase, T4 RNA ligase, T7 DNA ligase, Thermus thermophilus (Tth) ligase, Thermus aquaticus (Taq) ligase, or Pyrococcus furiosus (Pfu) ligase. Other internucleotide linkages include, without limitation, covalent bond formation between appropriate reactive groups such as between an α-haloacyl group and a phosphothioate group to form a thiophosphorylacetylamino group; and between a phosphorothioate and a tosylate or iodide group to form a 5′-phosphorothioester or pyrophosphate linkages.

In certain embodiments, chemical ligation may, under appropriate conditions, occur spontaneously such as by autoligation. Alternatively, in certain embodiments, “activating” or reducing agents may be used. Examples of activating agents and reducing agents include, without limitation, carbodiimide, cyanogen bromide (BrCN), imidazole, 1-methylimidazole/carbodiimide/cystamine, N-cyanoimidazole, dithiothreitol (DTT) and ultraviolet light. Nonenzymatic ligation according to certain embodiments may utilize specific reactive groups on the respective 3′ and 5′ ends of the aligned probes.

In certain embodiments, ligation generally comprises at least one cycle of ligation, for example, the sequential procedures of: hybridizing the target-specific portions of a first probe and a second probe, that are suitable for ligation, to their respective complementary regions on a target nucleic acid sequence; ligating the 3′ end of the first probe with the 5′ end of the second probe to form a ligation product; and denaturing the nucleic acid duplex to separate the ligation product from the target nucleic acid sequence. The cycle may or may not be repeated. For example, without limitation, thermocycling the ligation reaction can be employed to linearly increase the amount of ligation product.

According to certain embodiments, one may use ligation techniques such as gap-filling ligation, including, without limitation, gap-filling OLA and LCR, bridging oligonucleotide ligation, FEN-LCR, and correction ligation. Descriptions of these techniques can be found, among other places, in U.S. Pat. No. 5,185,243, published European Patent Applications EP 320308 and EP 439182, published PCT Patent Application WO 90/01069, published PCT Patent Application WO 02/02823, and U.S. patent application Ser. No. 09/898,323.

In certain embodiments, one forms a test composition for a subsequent amplification reaction by subjecting a ligation reaction composition to at least one cycle of ligation. In certain embodiments, after ligation, the test composition may be used directly in the subsequent amplification reaction. In certain embodiments, prior to the amplification reaction, the test composition may be subjected to a purification technique that results in a test composition that includes less than all of the components that may have been present after the at least one cycle of ligation. For example, in certain embodiments, one may purify the ligation product.

Purifying the ligation product according to certain embodiments comprises any process that removes at least some unligated probes, target nucleic acid sequences, enzymes, and/or accessory agents from the ligation reaction composition following at least one cycle of ligation. Such processes include, but are not limited to, molecular weight/size exclusion processes, e.g., gel filtration chromatography or dialysis, sequence-specific hybridization-based pullout methods, affinity capture techniques, precipitation, adsorption, or other nucleic acid purification techniques. The skilled artisan will appreciate that purifying the ligation product prior to amplification in certain embodiments reduces the quantity of primers needed to amplify the ligation product, thus reducing the cost of detecting a target sequence. Also, in certain embodiments, purifying the ligation product prior to amplification may decrease possible side reactions during amplification and may reduce competition from unligated probes during hybridization.

Hybridization-based pullout (HBP) according to certain embodiments of the present invention comprises a process wherein a nucleotide sequence complementary to at least a portion of one probe (or its complement), for example, the primer-specific portion, is bound or immobilized to a solid or particulate pullout support (see, e.g., U.S. Pat. No. 6,124,092). In certain embodiments, a composition comprising ligation product, target sequences, and unligated probes is exposed to the pullout support. The ligation product, under appropriate conditions, hybridizes with the support-bound sequences. In certain embodiments, the unbound components of the composition are removed, purifying the ligation products from those ligation reaction composition components that do not contain sequences complementary to the sequence on the pullout support. One subsequently removes the purified ligation products from the support and combines them with at least one primer set to form a first amplification reaction composition. The skilled artisan will appreciate that, in certain embodiments, additional cycles of HBP using different complementary sequences on the pullout support may remove all or substantially all of the unligated probes, further purifying the ligation product.

In certain embodiments, one may substantially remove certain unligated probes employing a probe set that includes a binding moiety on either the 5′ end of the first probe or the 3′ end of the second probe. In certain such embodiments, after a ligation reaction, one exposes the composition to a support that binds to the binding moiety. In certain embodiments, the unbound components of the composition are removed, substantially purifying the ligation products from those ligation reaction composition components that do not include the binding moiety, including the unligated probes without a binding moiety. In certain such embodiments, one may then remove the bound components from the support, and then expose them to a support with a bound sequence that is complementary to a portion of the ligation probe without the binding moiety, and that is not complementary to a portion of the ligation probe with the binding moiety. Thus, in certain such embodiments, the unligated first and second probes will be substantially removed from the ligation product. In certain embodiments, one may reverse the process by exposing the composition first to the support with the complementary sequence and second to the support that binds to the binding moiety. In certain embodiments, the binding moiety is biotin, which binds to streptavidin on the support.

In certain embodiments, one may employ different binding moieties (e.g., a first binding moiety and a second binding moiety) on the first probe and second probe of a probe set. In certain such embodiments, after a ligation reaction, one may then expose the composition to a first support that binds one of the binding moieties to capture ligation product and unligated probe with the first binding moiety. In certain embodiments, after removing unbound components, one may then remove the bound components and expose them to a second support that binds the second binding moiety to capture ligation product.

In certain embodiments, one may substantially remove unligated ligation probes using certain exonucleases that act specifically on single stranded nucleic acid. For example, in certain embodiments, one may employ a ligation probe set or sets that include a protective group on one end such that, when the ligation probes are ligated to one another, both ends of the ligation product will be protected from exonuclease digestion. In such embodiments, unligated probes are not protected on one end such that unligated probes are digested by exonuclease. In certain such embodiments, the 5′ end of the first probe includes a protective group, and the 3′ end of the second probe includes a protective group. One skilled in the art will appreciate certain exonucleases and certain protective groups that may be employed according to certain embodiments. In certain embodiments, biotin is used as a protective group. In certain embodiments, one may employ a method such that the exonuclease activity is substantially removed prior to an amplification reaction. In certain embodiments, one may employ an exonuclease that loses activity when exposed to a particular temperature for a given amount of time.

Amplification according to the present invention encompasses a broad range of techniques for amplifying nucleic acid sequences, either linearly or exponentially. Exemplary amplification techniques include, but are not limited to, PCR or any other method employing a primer extension step, and transcription or any other method of generating at least one RNA transcription product. Other nonlimiting examples of amplification are ligase detection reaction (LDR), and ligase chain reaction (LCR). Another nonlimiting exemplary amplification is a whole genome amplification. Amplification methods may comprise thermal-cycling or may be performed isothermally. The term “amplification product” includes products from any number of cycles of amplification reactions, primer extension reactions, and RNA transcription reactions, unless otherwise apparent from the context.

In certain embodiments, amplification methods comprise at least one cycle of amplification, for example, but not limited to, the sequential procedures of: hybridizing primers to primer-specific portions of the ligation product or amplification products from any number of cycles of an amplification reaction; synthesizing a strand of nucleotides in a template-dependent manner using a polymerase; and denaturing the newly-formed nucleic acid duplex to separate the strands. The cycle may or may not be repeated. In certain embodiments, amplification methods comprise at least one cycle of amplification, for example, but not limited to, the sequential procedures of: interaction of a polymerase with a promoter; synthesizing a strand of nucleotides in a template-dependent manner using a polymerase; and denaturing the newly-formed nucleic acid duplex to separate the strands. The cycle may or may not be repeated.

Descriptions of certain amplification techniques can be found, among other places, in H. Ehrlich et al., Science, 252:1643-50 (1991), M. Innis et al., PCR Protocols: A Guide to Methods and Applications, Academic Press, New York, N.Y. (1990), R. Favis et al., Nature Biotechnology 18:561-64 (2000), and H. F. Rabenau et al., Infection 28:97-102 (2000); Sambrook and Russell, Ausbel et al.

Primer extension according to the present invention is an amplification process comprising elongating a primer that is annealed to a template in the 5′ to 3′ direction using a template-dependent polymerase. According to certain embodiments, with appropriate buffers, salts, pH, temperature, and nucleotide triphosphates, including analogs and derivatives thereof, a template dependent polymerase incorporates nucleotides complementary to the template strand starting at the 3′-end of an annealed primer, to generate a complementary strand. Detailed descriptions of primer extension according to certain embodiments can be found, among other places in Sambrook et al., Sambrook and Russell, and Ausbel et al.

Transcription according to certain embodiments is an amplification process comprising an RNA polymerase interacting with a promoter on a single- or double-stranded template and generating a RNA polymer in a 5′ to 3′ direction. In certain embodiments, the transcription reaction composition further comprises transcription factors. RNA polymerases, including but not limited to T3, T7, and SP6 polymerases, according to certain embodiments, can interact with double-stranded promoters. Detailed descriptions of transcription according to certain embodiments can be found, among other places in Sambrook et al., Sambrook and Russell, and Ausbel et al.

Certain embodiments of amplification may employ multiplex amplification, in which multiple target sequences are simultaneously amplified (see, e.g., H. Geada et al., Forensic Sci. Int. 108:31-37 (2000) and D. G. Wang et al., Science 280:1077-82 (1998)).

In certain embodiments, one employs asymmetric PCR. According to certain embodiments, asymmetric PCR comprises an amplification reaction composition comprising (i) at least one primer set in which there is an excess of one primer (relative to the other primer in the primer set); (ii) at least one primer set that comprises only a first primer or only a second primer; (iii) at least one primer set that, during given amplification conditions, comprises a primer that results in amplification of one strand and comprises another primer that is disabled; or (iv) at least one primer set that meets the description of both (i) and (iii) above. Consequently, when the ligation product is amplified, an excess of one strand of the amplification product (relative to its complement) is generated.

In certain embodiments, one may use at least one primer set wherein the melting temperature (Tm₅₀) of one of the primers is higher than the Tm₅₀ of the other primer. Such embodiments have been called asynchronous PCR (A-PCR). See, e.g., U.S. patent application Ser. No. 09/875,211, filed Jun. 5, 2001. In certain embodiments, the Tm₅₀ of the first primer is at least 4-15° C. different from the Tm₅₀ of the second primer. In certain embodiments, the Tm₅₀ of the first primer is at least 8-15° C. different from the Tm₅₀ of the second primer. In certain embodiments, the Tm₅₀ of the first primer is at least 10-15° C. different from the Tm₅₀ of the second primer. In certain embodiments, the Tm₅₀ of the first primer is at least 10-12° C. different from the Tm₅₀ of the second primer. In certain embodiments, in at least one primer set, the Tm₅₀ of the at least one first primer differs from the melting temperature of the at least one second primer by at least about 4° C., by at least about 8° C., by at least about 10° C., or by at least about 12° C.

In certain embodiments of A-PCR, in addition to the difference in Tm₅₀ of the primers in a primer set, there is also an excess of one primer relative to the other primer in the primer set. In certain embodiments, there is a five to twenty-fold excess of one primer relative to the other primer in the primer set. In certain embodiments of A-PCR, the primer concentration is at least 50 nM.

In A-PCR according to certain embodiments, one may use conventional PCR in the first cycles such that both primers anneal and both strands are amplified. By raising the temperature in subsequent cycles, however, one may disable the primer with the lower Tₘ such that only one strand is amplified. Thus, the subsequent cycles of A-PCR in which the primer with the lower Tₘ is disabled result in asymmetric amplification. Consequently, when the ligation product is amplified, an excess of one strand of the amplification product (relative to its complement) is generated.

According to certain embodiments of A-PCR, the level of amplification can be controlled by changing the number of cycles during the first phase of conventional PCR cycling. In such embodiments, by changing the number of initial conventional cycles, one may vary the amount of the double strands that are subjected to the subsequent cycles of PCR at the higher temperature in which the primer with the lower Tₘ is disabled.

In certain embodiments, an A-PCR protocol may comprise use of a pair of primers, each of which has a concentration of at least 50 nM. In certain embodiments, conventional PCR, in which both primers result in amplification, is performed for the first 20-30 cycles. In certain embodiments, after 20-30 cycles of conventional PCR, the annealing temperature increases to 66-70° C., and PCR is performed for 5 to 40 cycles at the higher annealing temperature. In such embodiments, the lower Tₘ primer is disabled during such 5 to 40 cycles at higher annealing temperature. In such embodiments, asymmetric amplification occurs during the second phase of PCR cycles at a higher annealing temperature.

In certain embodiments, one employs asymmetric reamplification. According to certain embodiments, asymmetric reamplification comprises generating single-stranded amplification product in a second amplification process. In certain embodiments, the double-stranded amplification product of a first amplification process serves as the amplification target in the asymmetric reamplification process. In certain embodiments, one may achieve asymmetric reamplification using asynchronous PCR in which initial cycles of PCR conventionally amplify two strands and subsequent cycles are performed at a higher annealing temperature that disables one of the primers of a primer set as discussed above. In certain embodiments, the second amplification reaction composition comprises at least one primer set which comprises the at least one first primer, or the at least one second primer of a primer set, but typically not both. The skilled artisan understands that, in certain embodiments, asymmetric reamplification will also eventually occur if the primers in the primer set are not present in an equimolar ratio. In certain asymmetric reamplification methods, typically only single-stranded amplicons are generated since the second amplification reaction composition comprises only first or second primers from each primer set or a non-equimolar ratio of first and second primers from a primer set.

In certain embodiments, additional polymerase may also be a component of the second amplification reaction composition. In certain embodiments, there may be sufficient residual polymerase from the first amplification composition to synthesize the second amplification product.

Methods of optimizing amplification reactions are well known to those skilled in the art. For example, it is well known that PCR may be optimized by altering times and temperatures for annealing, polymerization, and denaturing, as well as changing the buffers, salts, and other reagents in the reaction composition. Optimization may also be affected by the design of the amplification primers used. For example, the length of the primers, as well as the G-C:A-T ratio may alter the efficiency of primer annealing, thus altering the amplification reaction. See James G. Wetmur, “Nucleic Acid Hybrids, Formation and Structure,” in Molecular Biology and Biotechnology, pp.605-8, (Robert A. Meyers ed., 1995).

In certain embodiments, different tag sequences are used to determine the probes that have been ligated. In certain embodiments, the tag sequences hybridize to particular capture oligonucleotides on a support.

In certain embodiments, different ligation and/or amplification products are detected by mobility discrimination using separation techniques. In certain embodiments, the tag sequences may have uniquely identifiable lengths or molecular weights. In certain embodiments, the tag sequence that corresponds to one target nucleic acid sequence is 2 nucleotides in length, the tag sequence that corresponds to a second target nucleic acid sequence is 4 nucleotides in length, the tag sequence that corresponds to a third target nucleic acid sequence is 6 nucleotides in length, and so forth. In certain embodiments the tag sequence is less than 101 nucleotides (i.e., 0 to 100 nucleotides) long, less than 41 nucleotides (i.e., 0 to 40 nucleotides) long, or 2 to 36 nucleotides long. In certain embodiments, the tag sequence that correspond to a particular target nucleic acid sequence will differ in length from the tag sequences that correspond to different target nucleic acid sequences by at least two nucleotides.

In certain embodiments, a tag sequence may comprise a sequence that is complementary to at least a portion of a particular mobility-modifier. In certain embodiments, a mobility modifier comprises (1) a tag complement for selectively hybridizing to the tag sequence of a ligation product and/or an amplification product, and (2) a tail portion for effecting a particular mobility in a mobility-dependent analysis technique (MDAT), e.g., electrophoresis, e.g., U.S. patent application No. 09/522,640, filed Mar. 15, 1999. Thus, in certain embodiments, ligation products and/or amplification products can be separated by molecular weight or length to determine the products present.

In certain embodiments, the detection of a ligation product and/or an amplification product in a particular molecular weight or length bin indicates the presence of the corresponding target nucleic acid sequence in the starting material. Descriptions of exemplary, but nonlimiting, mobility discrimination techniques may be found, among other places, in U.S. Pat. Nos. 5,470,705, 5,514,543, 5,580,732, 5,624,800, and 5,807,682.

In certain exemplary embodiments, air-dried ligation product and/or amplification product pellets, comprising ligation products and/or amplification products of uniquely identifiable molecular weight, are resuspended in buffer or deionized formamide. In certain embodiments, the resuspended samples and a molecular weight marker (e.g., GS 500 size standard, Applied Biosystems, Foster City, Calif.) are loaded onto an electrophoresis platform (e.g., ABI Prism™ Genetic Analyzer, Applied Biosystems) and electrophoresed in POP-4 polymer (Applied Biosystems) at 15 kV using a 50 μl capillary. In certain embodiments, the bands are detected or quantitated, and their position relative to the marker is determined. In certain embodiments, the bands are identified based on their relative electrophoretic mobility, indicating the presence of their respective target nucleic acid sequence in the sample. The bands may be quantitated, for example, based on the relative intensity of the associated label.

According to certain embodiments, certain tag sequences and tag complements should form a complex that (1) is stable under conditions typically used in nucleic acid analysis methods, e.g., aqueous, buffered solutions at room temperature; (2) is stable under mild nucleic-acid denaturing conditions; and (3) does not adversely effect the sequence specific binding of a target-specific portion of a probe with a target nucleic acid sequence. In certain embodiments, tag sequences and tag complements accommodate sets of distinguishable tag sequences and tag complements such that a plurality of different ligation products and/or amplification products and associated mobility modifiers may be present in the same reaction volume without causing unintended cross-interactions among the tag sequences, tag complements, target nucleic acid sequence, and target-specific portions of the probes. Certain methods for selecting sets of tag sequences that minimally cross hybridize are described, e.g., in Brenner and Albrecht, PCT Patent Application No. WO 96/41011.

In certain embodiments, the tag sequences and tag complements each comprise polynucleotides. In certain embodiments, the polynucleotide tag complements are rendered non-extendable by a polymerase, e.g., by including sugar modifications such as a 3′-phosphate, a 3′-acetyl, a 2′-3′-dideoxy, a 3′-amino, and a 2′-3′ dehydro.

In certain embodiments, a tag sequence and tag complement pair comprises a tag sequence that is a conventional synthetic polynucleotide, and a tag complement that is PNA. In certain embodiments, where the PNA tag complement has been-designed to form a triplex structure with a tag, the tag complement may include a “hinge” region in order to facilitate triplex binding between the tag sequence and tag complement. In certain embodiments, tag sequences and tag complement sequences comprise repeating sequences. Such repeating sequences in the tag sequences and tag complement are used in certain embodiments for their (1) high binding affinity, (2) high binding specificity, and (3) high solubility. An exemplary repeating sequence for use as a duplex-forming tag sequences or tag complement in certain embodiments is (CAG)ₙ, where the three base sequence is repeated from about 1 to 10 times (see, e.g., Boffa, et al., PNAS (USA), 92:1901-05 (1995); Wittung, et al., Biochemistry, 36:7973-79 (1997)). An exemplary repeating sequence for use as a triplex-forming tag sequences or tag complement in certain embodiments is (TCC)ₙ.

PNA and PNA/DNA chimera molecules can be synthesized using well known methods on commercially available, automated synthesizers, with commercially available reagents (see, e.g., Dueholm, et al., J. Org. Chem., 59:5767-73 (1994); Vinayak, et al., Nucleosides & Nucleotides, 16:1653-56 (1997)).

In certain embodiments, the tail portion of a mobility modifier may be any entity capable of effecting a particular mobility of a complex, such as all or a portion of a ligation product and/or an amplification product associated with the mobility modifier, in a mobility-dependent analysis technique. In certain embodiments, the tail portion of the mobility modifier may have one or more of the following characteristics: (1) have a low polydispersity in order to effect a well-defined and easily resolved mobility, e.g., Mw/Mn less than 1.05; (2) be soluble in an aqueous medium; (3) not adversely affect probe-target hybridization or tag sequence/tag complement hybridization; and (4) be available in sets such that members of different sets impart distinguishable mobilities to their associated complexes.

In certain embodiments, the tail portion of the mobility modifier comprises a polymer. In certain embodiments, the polymer may be homopolymer, random copolymer, or block copolymer. In certain embodiments, the polymer may have a linear, comb, branched, or dendritic architecture. In certain embodiments, mobility modifiers comprise more than one polymer chain element, where the elements collectively form a tail portion.

Exemplary polymers for use in the present invention include, but are not limited to, hydrophilic, or at least sufficiently hydrophilic when bound to a tag complement so that the tag complement is readily soluble in aqueous medium. In certain embodiments, where the mobility-dependent analysis technique is electrophoresis, the polymers are uncharged or have a charge/subunit density that is substantially less than that of the amplification product.

In certain embodiments, the polymer is polyethylene oxide (PEO), e.g., formed from one or more hexaethylene oxide (HEO) units, where the HEO units are joined end-to-end to form an unbroken chain of ethylene oxide subunits. Other exemplary embodiments include, but are not limited to, a chain composed of N 12mer PEO units, and a chain composed of N tetrapeptide units, where N is an adjustable integer (e.g., Grossman et al., U.S. Pat. No. 5,777,096).

In certain embodiments, coupling of the polymer tails to a polynucleotide tag complement can be carried out by an extension of conventional phosphoramidite polynucleotide synthesis methods, or by other standard coupling methods, e.g., a bis-urethane tolyl-linked polymer chain may be linked to a polynucleotide on a solid support via a phosphoramidite coupling. In certain embodiments, the polymer chain can be built up on a polynucleotide by stepwise addition of polymer-chain units to the polynucleotide, e.g., using standard solid-phase polymer synthesis methods.

In certain embodiments, the contribution of the tail to the mobility of the complex ligation product mobility modifier complex and/or amplification product mobility modifier complex, generally depends on the size of the tail. In certain embodiments, addition of charged groups to the tail, e.g., charged linking groups in the PEO chain, or charged amino acids in a polypeptide chain, may be used to achieve selected mobility characteristics. In certain embodiments, the mobility of a complex may be influenced by the properties of the ligation product and/or amplification product, e.g., in electrophoresis in a sieving medium, a larger probe in certain embodiments, may reduce the electrophoretic mobility of the complex comprising a mobility modifier.

When a tag complement is a polynucleotide, the tag complement may comprise all, part, or none of the tail portion of the mobility modifier. In certain embodiments of the invention, the tag complement may consist of some or all of the tail portion of the mobility modifier. In certain embodiments, the tag complement does not comprise any portion of the tail portion of the mobility modifier. For example, in certain embodiments, because PNA is uncharged, particularly when using free solution electrophoresis as the mobility-dependent analysis technique, the same PNA oligomer may act as both a tag complement and a tail portion of a mobility modifier.

In certain embodiments, the tag complement includes a hybridization enhancer, where, as used herein, the term “hybridization enhancer” means a moiety that serves to enhance, stabilize, or otherwise positively influence hybridization between two polynucleotides. Certain exemplary embodiments include, but are not limited to, intercalators (e.g., U.S. Pat. No. 4,835,263), minor-groove binders (e.g., U.S. Pat. No. 5,801,155), and cross-linking functional groups. In various embodiments, the hybridization enhancer may be attached to any portion of a mobility modifier. In certain embodiments, the hybridization enhancer is covalently attached to a mobility modifier. In certain embodiments, a hybridization enhancer is minor-groove binder, e.g., but not limited to, netropsin, distamycin, and the like.

In certain embodiments, a plurality of ligation product/mobility modifier complexes and/or amplification product/mobility modifier complexes are resolved via a MDAT.

In certain embodiments, ligation product/mobility modifier complexes and/or amplification product/mobility modifier complexes are resolved (separated) by liquid chromatography. Exemplary stationary phase media for use in certain exemplary methods include reversed-phase media (e.g., C-18 or C-8 solid phases), ion-exchange media (particularly anion-exchange media), and hydrophobic interaction media. In certain embodiments, the ligation product/mobility modifier complexes and/or amplification product/mobility modifier complexes are separated by micellar electrokinetic capillary chromatography (MECC).

In certain embodiments, reversed-phase chromatography is carried out using an isocratic, or a linear, curved, or stepped solvent gradient, wherein the level of a nonpolar solvent such as acetonitrile or isopropanol in aqueous solvent is increased during a chromatographic run, causing analytes to elute sequentially according to affinity of each analyte for the solid phase. In certain embodiments, for separating polynucleotides, an ion-pairing agent (e.g., a tetra-alkylammonium) is included in the solvent to mask the charge of phosphate.

According to certain embodiments, the ligation product/mobility modifier complexes and/or amplification product/mobility modifier complexes are resolved by electrophoresis in a sieving or non-sieving matrix and quantitated. In certain embodiments, the electrophoretic separation is carried out in a capillary tube by capillary electrophoresis (see, e.g., Capillary Electrophoresis: Theory and Practice, Grossman and Colburn eds., Academic Press (1992)). Sieving matrices that may be used include, but are not limited to, covalently crosslinked matrices, such as polyacrylamide covalently crosslinked with bis-acrylamide; gel matrices formed with linear polymers (e.g., Madabhushi et al., U.S. Pat. No. 5,552,028); and gel-free sieving media (e.g., Grossman et al., U.S. Pat. No. 5,624,800; Hubert and Slater, Electrophoresis, 16: 2137-2142 (1995); Mayer et al., Analytical Chemistry, 66(10): 1777-1780 (1994)). In certain embodiments, the electrophoresis medium may contain a nucleic acid denaturant, such as 7M formamide, for maintaining polynucleotides in single-stranded form. Suitable capillary electrophoresis instrumentation are commercially available, e.g., the ABI PRISM™ Genetic Analyzer (Applied Biosystems).

In certain embodiments, following at least one amplification cycle, the amplification products are separated based on their molecular weight or length or mobility by, for example, without limitation, gel electrophoresis, HPLC, MALDI-TOF, gel filtration, or mass spectroscopy. In certain embodiments, the detection and quantitation of a labeled sequence at a particular mobility address indicates that the sample or starting material contains the corresponding target nucleic acid sequence.

In certain embodiments, tag sequences interact with particular beads that comprise a tag complement. See e.g., U.S. patent application Ser. Nos. 60/332,519, filed Nov. 21, 2001, and 60/384,731, filed May 31, 2002.

In certain embodiments, tag sequences interact with particular labeled probes that include a tag complement. See, e.g., U.S. patent application No. 60/412,225, filed Sep. 19, 2002.

In certain embodiments, ligation products and/or amplification products are detected using a double-stranded dependent label, such as an intercalating dye, e.g., as disclosed in U.S. patent application Ser. No. 60/421,035, filed Oct. 23, 2002.

### D. Certain Exemplary Embodiments of Detecting Targets

The present invention is directed to methods, reagents, and kits for determining the methylation state of a target cytosine. In certain embodiments, one employs a ligation reaction that results in a given ligation product only if a particular target nucleic acid sequence comprising a target cytosine having a particular methylation state is present in a sample. In certain embodiments, ligation products may form even if the appropriate target nucleic acid sequence comprising the target cytosine having the appropriate methylation state is not in the sample, but such ligation occurs to a measurably lesser extent than when the appropriate target nucleic acid sequence is in the sample. Exemplary ligation reactions, include, but are not limited to, those discussed in U.S. Pat. No. 6,027,889, Published PCT Patent Application No. WO 01/92579, published PCT Patent Application WO 97/31256, and U.S. patent application Ser. Nos. 09/584,905 and 10/011,993.

In certain such embodiments, for each target nucleic acid sequence to be detected, one forms a ligation reaction composition comprising a probe set, comprising at least one first probe and at least one second probe. In certain embodiments, the probe set is subjected to at least one cycle of ligation, wherein adjacently hybridized first and second probes are ligated together to form a ligation product only if the particular target nucleic acid sequence comprising a target cytosine having a particular methylation state is present in the sample. In certain embodiments, ligation products may form even if the appropriate target nucleic acid sequence comprising the target cytosine having the appropriate methylation state is not in the sample, but such ligation occurs to a measurably lesser extent than when the appropriate target nucleic acid sequence is in the sample.

In certain embodiments, the methylation state-influenced ligation is accomplished by a selective ligase. In certain embodiments, such a ligase results in selective ligation of adjacently hybridized probes of a probe set if the target cytosine is methylated. In certain embodiments, such a ligase results in selective ligation of adjacently hybridized probes of a probe set if the target cytosine is not methylated. The term “selective ligation” means that ligation occurs to a measurably lesser extent in the presence of target nucleic acid sequences that do not have a target cytosine in the appropriate methylation state than in the presence of target nucleic acid sequences that have a target cytosine with the appropriate methylation state. In certain embodiments, ligation only occurs in the presence of target nucleic acid sequences that have a target cytosine in the appropriate methylation state.

In certain embodiments, the sample is first incubated with an agent that selectively modifies the target cytosine, depending on the methylation state of the target cytosine. The term “selectively modifies” means that modification of target cytosines occurs to a measurably lesser extent with target cytosines that do not have the appropriate methylation state than with target cytosines that have the appropriate methylation state. In certain embodiments, modification only occurs with target nucleic acid sequences that have a target cytosine in the appropriate methylation state. In certain embodiments, a modifying agent selectively binds to methylated cytosines. In certain other embodiments, a modifying agent selectively binds to unmethylated cytosines. The term “selectively binds” means that binding of target cytosines occurs to a measurably lesser extent with target cytosines that do not have the appropriate methylation state than with target cytosines that have the appropriate methylation state. In certain embodiments, binding only occurs in the presence of target nucleic acid sequences that have a target cytosine in the appropriate methylation state. In certain embodiments, the ligation reaction is affected by the presence or absence of a bound modifying agent. For example, in certain embodiments, ligation occurs to a measurably lesser extent if target nucleic acid sequence having bound modifying agent is present. In certain embodiments, ligation occurs to a measurably lesser extent if target nucleic acid sequence having bound modifying agent is absent. In certain embodiments, ligation only occurs if target nucleic acid sequence having bound modifying agent is present. In certain embodiments, ligation only occurs if target nucleic acid sequence having bound modifying agent is absent

In certain embodiments, the modifying agent selectively, chemically alters a target cytosine, depending on the methylation state of the target cytosine. The term “selectively, chemically alters” means that chemical alteration of target cytosines occurs to a measurably lesser extent with target cytosines that do not have the appropriate methylation state than with target cytosines that have the appropriate methylation state. In certain embodiments, chemical alteration only occurs in the presence of target nucleic acid sequences that have a target cytosine in the appropriate methylation state. In certain embodiments, a modifying agent selectively, chemically alters methylated cytosines. In certain other embodiments, a modifying agent selectively, chemically alters unmethylated cytosines. In certain embodiments, the ligation reaction is affected by the presence or absence of a chemically altered cytosine. In certain embodiments, ligation occurs to a measurably lesser extent if target nucleic acid sequence comprising chemically altered cytosine is present. In certain embodiments, ligation occurs to a measurably lesser extent if target nucleic acid sequence comprising chemically altered cytosine is absent. In certain embodiments, ligation only occurs if target nucleic acid sequence comprising chemically altered cytosine is absent. In certain embodiments, ligation only occurs if target nucleic acid sequence comprising chemically altered cytosine is absent.

In certain embodiments, the modifying agent selectively converts a target cytosine to a converted nucleotide, depending on the methylation state of the target cytosine. The term “selectively converts” means that conversion of target cytosines occurs to a measurably lesser extent with target cytosines that do not have the appropriate methylation state than with target cytosines that have the appropriate methylation state. In certain embodiments, conversion only occurs in the presence of target nucleic acid sequences that have a target cytosine in the appropriate methylation state. In certain embodiments, a modifying agent selectively converts methylated cytosines to converted nucleotides. In certain embodiments, a modifying agent selectively converts unmethylated cytosines to converted nucleotides. In certain embodiments, at least one probe set may be used to differentiate between targets with cytosines and targets with converted nucleotides.

In certain embodiments, bisulfite is employed as a modifying agent. See, e.g., U.S. Pat. No. 6,265,171; U.S. Pat No. 6,331,393. Incubating target nucleic acid sequence with bisulfite results in deamination of a substantial portion of unmethylated cytosines, which converts such cytosines to uracil. Methylated cytosines are deaminated to a measurably lesser extent. In certain embodiments, the sample is then amplified or replicated, resulting in the uracil bases being replaced with thymine. Thus, in certain embodiments, a substantial portion of unmethylated target cytosines ultimately become thymines, while a substantial portion of methylated cytosines remain cytosines. In certain embodiments, the identity of the nucleotide (cytosine, uracil, or thymine) of the target may be determined by a ligation assay. In certain embodiments, the identity of the nucleotide (cytosine, uracil, or thymine) of the target may be determined by a ligation and amplification assay.

In certain embodiments, other modifying agents may be used. In certain embodiments, the modifying agent need not catalyze deamination reactions and the converted nucleotide need not be uracil or thymine. Certain embodiments may employ any agent that is capable of selectively converting either methylated cytosines or unmethylated cytosines to another nucleotide.

As discussed above, certain embodiments employ a modifying agent that selectively converts either methylated or unmethylated target cytosines of a target nucleic acid sequence to a converted nucleotide. In certain such embodiments, after incubation with the modifying agent, the target cytosine or converted nucleotide is called a pivotal nucleotide, and the target nucleic acid sequence that has been incubated is called the test target nucleic acid sequence. In certain embodiments, the nucleotide of a probe that hybridizes to the pivotal nucleotide is called the pivotal complement. In certain such embodiments, one forms a test composition comprising at least one test target nucleic acid sequence by incubating the at least one target nucleic acid sequence with the modifying agent to selectively convert either methylated cytosine or unmethylated cytosine to a converted nucleotide.

In certain embodiments, one forms a ligation reaction composition comprising the test composition and a ligation probe set for each target nucleic acid sequence. In certain embodiments, the probe set comprises at least one first probe comprising a target specific portion, and at least one second probe comprising a target specific portion. In certain embodiments, at least one of the at least one first probe and the at least one second probe comprises at least one pivotal complement. In certain embodiments, the pivotal complement is complementary to the target cytosine. In certain embodiments, the pivotal complement is complementary to the converted nucleotide. In certain embodiments, one may employ two different first probes or two different second probes that comprise pivotal complements, namely, one pivotal complement that is complementary to cytosine and another pivotal complement that is complementary to the converted nucleotide.

In certain embodiments, at least one of the at least one first probe and the at least one second probe comprises a label. In certain embodiments, a probe comprising a label further comprises a pivotal complement. In certain embodiments, a probe comprising a label further comprises a pivotal complement that is complementary to cytosine. In certain embodiments, such a probe may be used to detect the presence or absence of a target cytosine that has not been converted. In certain embodiments, a probe comprising a label further comprises a pivotal complement that is complementary to the converted nucleotide. In certain embodiments, such a probe may be used to detect the presence or absence of a target cytosine that has been converted.

In certain embodiments, at least one of the at least one first probes comprises a label and a pivotal complement that is complementary to cytosine; and at least one of the at least one first probes comprises a different label and a pivotal complement that is complementary to the converted nucleotide. In certain such embodiments, the different labels provide detectably different signals.

In certain embodiments, at least one of the at least one first probes comprises a label and at least one of the at least one second probe comprises a pivotal complement. In certain such embodiments, the second probe comprises a pivotal complement that is complementary to the target cytosine. In certain other embodiments, the second probe comprises a pivotal complement that is complementary to the converted nucleotide. In certain such embodiments, the ligation reaction composition is subjected to at least one cycle of ligation. In certain embodiments, after at least one cycle of ligation, the methylation state of the target cytosine may be determined by detecting the presence or absence of ligation product comprising the label by separating ligation product from unligated probes based on size difference between the ligation product and unligated probe.

In certain embodiments, at least one probe comprises a tag sequence. In certain embodiments, tag sequences may be used in various combinations with labels to determine the methylation state of one or more target cytosines at one or more loci. One skilled in the art will readily understand that one or more different tag sequences, labels, and pivotal nucleotides may be used in various combinations on the same and/or on different first probes and/or on the same and/or different second probes. Certain non-limiting examples are provided.

In certain embodiments, either methylated cytosine is converted to a converted nucleotide, or unmethylated cytosine is converted to a converted nucleotide. In certain such embodiments, at least one of the at least one first probes comprises a tag sequence corresponding to a target cytosine at one locus; and at least one other of the at least one first probes comprises a tag sequence corresponding to a different target cytosine at a different locus. In certain embodiments, at least one of the at least one second probes comprises (1) a pivotal complement that is complementary to cytosine, and (2) a label. In certain embodiments, after at least one cycle of ligation, the tag sequences and the labels may be used to detect the presence or absence of ligation products to determine the methylation states of target cytosines at the different loci.

In certain embodiments, either methylated cytosine is converted to a converted nucleotide, or unmethylated cytosine is converted to a converted nucleotide. In certain such embodiments, at least one of the at least one first probes comprises a tag sequence corresponding to a target cytosine at one locus; and at least one other of the at least one first probes comprises a tag sequence corresponding to a different target cytosine at a different locus. In certain embodiments, at least one of the at least one second probes comprises (1) a pivotal complement that is complementary to the converted nucleotide, and (2) a label. In certain embodiments, after at least one cycle of ligation, the tag sequences and the labels may be used to detect the presence or absence of ligation products to determine the methylation states of target cytosines at the different loci.

In certain embodiments, either methylated cytosine is converted to a converted nucleotide, or unmethylated cytosine is converted to a converted nucleotide. In certain such embodiments, at least one of the at least one first probes comprises a tag sequence corresponding to a target cytosine at one locus; and at least one other of the at least one first probes comprises a tag sequence corresponding to a different target cytosine at a different locus. In certain embodiments, at least one of the at least one second probes comprises (1) a pivotal complement that is complementary to cytosine, and (2) a label; and at least one other of the at least one second probes comprises (1) a pivotal complement that is complementary to the converted nucleotide and (2) a detectably different label. In certain embodiments, after at least one cycle of ligation, the tag sequences and the labels may be used to detect the presence or absence of ligation products to determine the methylation states of target cytosines at the different loci.

In certain embodiments, either methylated cytosine is converted to a converted nucleotide, or unmethylated cytosine is converted to a converted nucleotide. In certain such embodiments, at least one of the at least one first probes comprises (1) a tag sequence specific for a target cytosine at one locus, and (2) a pivotal complement that is complementary to cytosine; and at least one other of the at least one first probes comprises (1) a tag sequence specific for a different target cytosine at a different locus and (2) a pivotal complement that is complementary to cytosine. In certain embodiments, at least one of the at least one second probes comprises a label. In certain embodiments, after at least one cycle of ligation, the tag sequences and the labels may be used to detect the presence or absence of ligation products to determine the methylation states of target cytosines at the different loci.

In certain embodiments, either methylated cytosine is converted to a converted nucleotide, or unmethylated cytosine is converted to a converted nucleotide. In certain such embodiments, at least one of the at least one first probes comprises (1) a tag sequence specific for a target cytosine at one locus, and (2) a pivotal complement that is complementary to the converted nucleotide; and at least one other of the at least one first probes comprises (1) a tag sequence specific for a different target cytosine at a different locus and (2) a pivotal complement that is complementary to the converted nucleotide. In certain embodiments, at least one of the at least one second probes comprises a label. In certain embodiments, after at least one cycle of ligation, the tag sequences and the labels may be used to detect the presence or absence of ligation products to determine the methylation states of target cytosines at the different loci.

In certain embodiments, the first and second target-specific probes in each probe set are designed to be complementary to the sequences immediately flanking a pivotal nucleotide of a target nucleic acid sequence. In certain embodiments, either the first target-specific probe or the second target-specific probe of a probe set, but not both, will comprise the pivotal complement. When the target nucleic acid sequence is present in the sample, the first and second target-specific probes will hybridize, under appropriate conditions, to adjacent regions on the target. When the pivotal complement is base-paired in the presence of an appropriate ligation agent, two adjacently hybridized probes may be ligated together to form a ligation product. In certain embodiments, one can then detect the presence or absence of the target nucleic acid sequences by detecting the presence or absence of the ligation product

According to certain embodiments, the first and second probes in each ligation probe set are designed to be complementary to the sequences immediately flanking the pivotal nucleotide of the target sequence (see, e.g., probes A, B, and Z in FIG. 5(A)). In the embodiment shown in FIG. 5, two first probes A and B of a ligation probe set comprise a different nucleotide at the pivotal complement and a different label (LBA or LBB) for each different nucleotide at the pivotal complement. In the embodiment shown in FIG. 5, the second probe Z of the ligation probe set comprises a tag sequence 10 that corresponds to the locus being analyzed. Thus, in certain embodiments, multiple probe sets may be used to determine the methylation state of multiple target cytosines at multiple different loci. In certain such embodiments, one may employ multiple probe sets that each include: first probes that may be used to determine the methylation state of different target cytosines in view of labels; and different second probes that may be used to separate ligation products for the different loci being analyzed using the different tag sequences. One forms a ligation reaction composition comprising the probe set and the sample.

When the target sequence is present in the sample, the first and second probes will hybridize, under appropriate conditions, to adjacent regions on the target (see, e.g., FIG. 5(B)). When the pivotal complement is base-paired to the target, in the presence of an appropriate ligation agent, two adjacently hybridized probes may be ligated together to form a ligation product (see, e.g., FIG. 5(C)). If the pivotal complement of a first probe is not base-paired to the target, no ligation product comprising that mismatched probe will be formed (see, e.g., probe B in FIGS. 5(B) to 5(D). In certain embodiments, ligation of probes with a pivotal complement that is not complementary to the pivotal nucleotide may occur, but such ligation occurs to a measurably lesser extent than ligation of probes with a pivotal complement that is complementary to the pivotal nucleotide.

In the example shown in FIG. 5, a mismatched base at the pivotal nucleotide interferes with ligation, even if both probes are otherwise fully hybridized to their respective target regions. In certain embodiments, other mechanisms may be employed to avoid ligation of probes that do not include the correct complementary nucleotide at the pivotal complement. For example, in certain embodiments, conditions may be employed such that a probe of a ligation probe set will hybridize to the target sequence to a measurably lesser extent if there is a mismatch at the pivotal nucleotide. Thus, in such embodiments, such non-hybridized probes will not be ligated to the other probe in the probe set.

In certain embodiments, the first probes and second probes in a ligation probe set are designed with similar melting temperatures (Tₘ). Where a probe includes a pivotal complement, in certain embodiments, the Tₘ for the probe(s) comprising the pivotal complement(s) of the target pivotal nucleotide sought will be approximately 4-15° C. lower than the other probe(s) that do not contain the pivotal complement in the probe set. In certain such embodiments, the probe comprising the pivotal complement(s) will also be designed with a Tₘ near the ligation temperature. Thus, a probe with a mismatched nucleotide will more readily dissociate from the target at the ligation temperature. The ligation temperature, therefore, in certain embodiments provides another way to discriminate between, for example, multiple potential alleles in the target.

Further, in certain embodiments, ligation probe sets do not comprise a pivotal complement at the terminus of the first or the second probe (e.g., at the 3′ end or the 5′ end of the first or second probe). Rather, the pivotal complement is located somewhere between the 5′ end and the 3′ end of the first or second probe. In certain such embodiments, probes with target-specific portions that are fully complementary with their respective target regions will hybridize under high stringency conditions. Probes with one or more mismatched bases in the target-specific portion, by contrast, will hybridize to their respective target region to a measurably lesser extent. Both the first probe and the second probe must be hybridized to the target for a ligation product to be generated.

In certain embodiments as shown in FIG. 5(E), after ligation of appropriately hybridized first and second probes, the ligation products may be detected by hybridization of the tag sequences 10 of the ligation products to capture oligonucleotides 10′ on an addressable support. In certain embodiments, one may determine the pivotal nucleotide of the target nucleic acid sequence in view of the label. In certain embodiments, the tag sequence may be a mobility modifier, which allows separation of ligation products with different tag sequences by a mobility dependent analysis technique. In certain embodiments, the tag sequences may be hybridized to appropriate mobility modifiers and then the presence of particular ligation products may be detected using a mobility dependent analysis technique.

FIG. 3 shows certain embodiments in which one employs a probe set that is similar to the probe set shown in FIG. 5, but in which the two first probes A and B of a ligation probe set comprise a different nucleotide at the pivotal complement and a different tag sequence 12 and 14, respectively, for each different nucleotide at the pivotal complement. In the embodiment shown in FIG. 3, the second probe Z of the ligation probe set comprises a label (LZ) that corresponds to the locus being analyzed. Thus, in certain embodiments, multiple probe sets may be used to determine the methylation state of different target cytosines at multiple different loci. In certain such embodiments, one may employ multiple probe sets that each include: different first probes that may be used to separate target nucleic acid sequences with different pivotal nucleotides using the different tag sequences; and different second probes that may be used to distinguish between different loci being analyzed in view of the different labels.

In certain embodiments, one may subject the initial sample to an amplification reaction to increase the amount of target nucleic acid to which probes in a probe set will hybridize.

In certain embodiments, the ligation reaction composition may comprise different probe sets for determining the methylation state of multiple different target cytosines at multiple loci. In certain embodiments, one may use, for example, without limitation, a screening assay to determine the methylation state of three different target cytosines at three different loci (e.g., L1, L2, and L3) using six probe sets. See, e.g., Table 1 below.

TABLE 1

| Locus | Meth. State | Probe Set - Probe |
|---|---|---|
| L1 | 1 Meth | A (red) - Z (tag 1) |
| | 2 Unmeth | B (blue) - Z (tag 1) |
| L2 | 1 Meth | C (red) - Y (tag 2) |
| 2 Unmeth | D (blue) - Y (tag 2) | |
| L3 | 1 Meth | E (red) - X (tag 3) |
| 2 Unmeth | F (blue) - X (tag 3) | |

In such embodiments, two different probe sets are used to detect the methylation state of each target cytosine at each locus. The two first target-specific probes of the two different probe sets for each locus, for example, probes A and B for locus L1, comprise the same upstream sequence-specific portion, but differ at the pivotal complement. Also, the two different probes A and B comprise different labels. The two second target-specific probes of the two different probe sets for each locus, for example, probe Z for locus L1, comprise the same downstream sequence-specific portion. Also, the probes Z comprise the same tag sequence. The tag sequence of the second target-specific probe for each different locus is different. Thus, in certain embodiments, each different tag sequence may be used to separate different ligation products for different loci from one another. In certain embodiments, both combinations of a probe set, such as AZ and BZ of the probe set for locus L1, will have the same tag sequence, and thus both methylation states for a given locus may be detected at the same position. Therefore, the label for target cytosines of locus L1 will be detected at position 1, the labels for target cytosines of locus L2 will be detected at position 2, and the labels for target cytosines of locus L3 will be detected at position 3.

Thus, in embodiments as depicted in Table 1, three probes A, B, and Z, are used to form the two possible L1 ligation products, wherein AZ is the ligation product for a methylated cytosine and BZ is the ligation product for an unmethylated cytosine. Likewise, probes C, D, and Y, are used to form the two possible L2 ligation products. Likewise, probes E, F, and X, are used to form the two possible L3 ligation products.

After ligation of adjacently hybridized first and second target-specific probes, in certain embodiments, one can detect the presence or absence of a ligation product for each methylation state of a target cytosine for each of the loci by using the unique combinations of labels and tag sequences. For example, in certain embodiments, one may determine the methylation state of a target cytosine by the label or labels detected at position 1 in view of tag sequence 1, determine the methylation state of a target cytosine by the label or labels detected at position 2 in view of tag sequence 2, and determine the methylation state of a target cytosine by the label or labels detected at position 3 in view of tag sequence 3. For example, a sample may result in a red label at position 1, a blue label at position 2, and both red and blue labels at position 3. One may conclude that such a sample includes methylated cytosines at locus L1, unmethylated cytosines at locus L2, and both methylated and unmethylated cytosines at locus L3.

The skilled artisan will understand that in certain embodiments, the probes can be designed with the pivotal complement at any location in either the first target-specific probe or the second target-specific probe. Additionally, in certain embodiments, target-specific probes comprising multiple pivotal complements are within the scope of the invention.

### Oligonucleotide Ligation and Amplification

In certain embodiments, one employs a ligation reaction followed by amplification to obtain analyte polynucleotides to detect target nucleic acids. Certain nonlimiting examples are shown in FIGS. 2 and 4.

According to certain embodiments, the first and second probes in each ligation probe set are designed to be complementary to the sequences immediately flanking the pivotal nucleotide of the target sequence (see, e.g., probes A, B, and Z in FIG. 4(A)). In the embodiment shown in FIG. 4, two first probes A and B of a ligation probe set comprise a different nucleotide at the pivotal complement and a different 5′ primer-specific portion (A1 and B1, respectively) for each different nucleotide at the pivotal complement. In the embodiment shown in FIG. 4, the second probe Z of the ligation probe set comprises a tag sequence 16 that corresponds to the locus being analyzed and a 3′ primer-specific portion Z1. Thus, in certain embodiments, multiple probe sets may be used to determine the methylation state of target cytosines at multiple different loci. In certain such embodiments, one may employ multiple probe sets that each include: different first probes that may be used to determine different methylation states of target cytosines in view of the different 5′ primer-specific portions; and different second probes that may be used to separate ligation products for the different loci being analyzed using the different tag sequences. One forms a ligation reaction composition comprising the probe set and the sample.

When the target sequence is present in the sample, the first and second probes will hybridize, under appropriate conditions, to adjacent regions on the target (see, e.g., FIG. 4(B)). When the pivotal complement is base-paired to the target, in the presence of an appropriate ligation agent, two adjacently hybridized probes may be ligated together to form a ligation product (see, e.g., FIG. 4(C)). If the pivotal complement of a first probe is not base-paired to the target, no ligation product comprising that mismatched probe will be formed (see, e.g., probe B in FIGS. 4(B) to 4(D). In certain embodiments, ligation of probes with a pivotal complement that is not complementary to the pivotal nucleotide may occur, but such ligation occurs to a measurably lesser extent than ligation of probes with a pivotal complement that is complementary to the pivotal nucleotide.

In the example shown in FIG. 4, a mismatched base at the pivotal nucleotide interferes with ligation, even if both probes are otherwise fully hybridized to their respective target regions. In certain embodiments, other mechanisms may be employed to avoid ligation of probes that do not include the correct complementary nucleotide at the pivotal complement. For example, in certain embodiments, conditions may be employed such that a probe of a ligation probe set will hybridize to the target sequence to a measurably lesser extent if there is a mismatch at the pivotal nucleotide. Thus, in such embodiments, such non-hybridized probes will not be ligated to the other probe in the probe set.

In certain embodiments, the first probes and second probes in a ligation probe set are designed with similar melting temperatures (Tₘ). Where a probe includes a pivotal complement, in certain embodiments, the Tₘ for the probe(s) comprising the pivotal complement(s) of the target pivotal nucleotide sought will be approximately 4-15° C. lower than the other probe(s) that do not contain the pivotal complement in the probe set. In certain such embodiments, the probe comprising the pivotal complement(s) will also be designed with a Tₘ near the ligation temperature. Thus, a probe with a mismatched nucleotide will more readily dissociate from the target at the ligation temperature. The ligation temperature, therefore, in certain embodiments provides another way to discriminate between, for example, multiple potential alleles in the target.

Further, in certain embodiments, ligation probe sets do not comprise a pivotal complement at the terminus of the first or the second probe (e.g., at the 3′ end or the 5′ end of the first or second probe). Rather, the pivotal complement is located somewhere between the 5′ end and the 3′ end of the first or second probe. In certain such embodiments, probes with target-specific portions that are fully complementary with their respective target regions will hybridize under high stringency conditions. Probes with one or more mismatched bases in the target-specific portion, by contrast, will hybridize to their respective target region to a measurably lesser extent. Both the first probe and the second probe must be hybridized to the target for a ligation product to be generated.

In certain embodiments, the ligation reaction composition (in the appropriate salts, buffers, and nucleotide triphosphates) is then combined with at least one primer set and a polymerase to form a first amplification reaction composition (see, e.g., FIG. 4(E)). In the first amplification cycle, the second primer, comprising a sequence complementary to the 3′ primer-specific portion of the ligation product, hybridizes with the ligation product and is extended in a template-dependent fashion to create a double-stranded molecule comprising the ligation product and its complement (see, e.g., FIGS. 4(E)-(F)). Subsequent amplification cycles may exponentially amplify this double-stranded molecule (see, e.g., FIGS. 4(F)-(J)). In FIG. 4, for example, primers PA* and PB* include different labels. Thus, amplification products resulting from incorporation of these primers will include a label specific for the particular pivotal nucleotide that is included in the original target sequence.

In certain embodiments as shown in FIG. 4(K), after ligation of appropriately hybridized first and second probes, the ligation products may be detected by hybridization of the tag sequences 16 of the ligation products to capture oligonucleotides on an addressable support. In certain embodiments, one may determine the pivotal nucleotide of the target nucleic acid sequence in view of the label. In certain embodiments, the tag sequence may be a mobility modifier, which allows separation of ligation products with different tag sequences by a mobility dependent analysis technique. In certain embodiments, the tag sequences may be hybridized to appropriate mobility modifiers and then the presence of particular ligation products may be detected using a mobility dependent analysis technique.

FIG. 2 shows certain embodiments in which one employs a probe set that is similar to the probe set shown in FIG. 4, but in which the two first probes A and B of a ligation probe set comprise a 5′ primer-specific portion PS, a different nucleotide at the pivotal complement, and a different tag sequence (18 or 20) for each different nucleotide at the pivotal complement. In the embodiment shown in FIG. 2, the second probe Z of the ligation probe set comprises a 3′ primer-specific portion Z1 that corresponds to the locus being analyzed.

FIGS. 2(E) to (F) shows certain embodiments in which amplification involves primers PX and PZ. In the embodiments shown in FIG. 2, primer PZ in includes a label corresponding to the locus being analyzed. Thus, in certain embodiments, multiple probe sets may be used to determine the methylation state of different target cytosines at multiple different loci. In certain such embodiments, one may employ multiple probe sets that each include: different first probes that may be used to separate target nucleic acid sequences with different pivotal nucleotides using the different tag sequences (or complements); and different primers that may be used to distinguish between different loci being analyzed in view of the different labels.

### E. Certain Exemplary Kits

In certain embodiments, the invention also provides kits designed to expedite performing certain methods. In certain embodiments, kits serve to expedite the performance of the methods of interest by assembling two or more components used in carrying out the methods. In certain embodiments, kits may contain components in pre-measured unit amounts to minimize the need for measurements by end-users. In certain embodiments, kits may include instructions for performing one or more methods of the invention. In certain embodiments, the kit components are optimized to operate in conjunction with one another.

In certain embodiments kits for determining the methylation state of a target cytosine in at least one target nucleic acid sequence in a sample comprise: a modifying agent that modifies methylated cytosine, but does not modify unmethylated cytosine; and a ligation probe set for each target cytosine, the probe set comprising: (a) at least one first probe, comprising a target-specific portion, and (b) at least one second probe, comprising a target-specific portion, wherein the probes in each set are suitable for ligation together when hybridized adjacent to one another on a complementary target nucleic acid sequence.

In certain embodiments, the at least one first probe further comprises a 5′ primer-specific portion, wherein the 5′ primer-specific-portion comprises a sequence, and the at least one second probe further comprises a 3′ primer-specific portion, wherein the 3′ primer-specific portion comprises a sequence. In certain embodiments, the kit further comprises: at least one primer set, the primer set comprising (i) at least one first primer comprising the sequence of the 5′ primer-specific portion of the first probe, and (ii) at least one second primer comprising a sequence complementary to the sequence of the 3′ primer-specific portion of the second probe.

In certain embodiments, kits for determining the methylation state of a target cytosine in at least one target nucleic acid sequence in a sample comprise: a modifying agent that modifies unmethylated cytosine, but does not modify methylated cytosine; and a ligation probe set for each target cytosine, the probe set comprising: (a) at least one first probe, comprising a target-specific portion, and (b) at least one second probe, comprising a target-specific portion, wherein the probes in each set are suitable for ligation together when hybridized adjacent to one another on a complementary target nucleic acid sequence.

In certain embodiments, the at least one first probe further comprises a 5′ primer-specific portion, wherein the 5′ primer-specific-portion comprises a sequence, and the at least one second probe further comprises a 3′ primer-specific portion, wherein the 3′ primer-specific portion comprises a sequence. In certain embodiments, the kit further comprises: at least one primer set, the primer set comprising (i) at least one first primer comprising the sequence of the 5′ primer-specific portion of the first probe, and (ii) at least one second primer comprising a sequence complementary to the sequence of the 3′ primer-specific portion of the second probe.

In certain embodiments, kits for determining the methylation state of a target cytosine in at least one target nucleic acid sequence in a sample comprise: a ligation probe set for each target cytosine, the probe set comprising: (a) at least one first probe, comprising a target-specific portion, and (b) at least one second probe, comprising a target-specific portion, wherein the probes in each set are suitable for ligation together when hybridized adjacent to one another on a complementary target nucleic acid sequence; and a selective ligase that ligates together adjacently hybridized probes only if the target cytosine is methylated.

In certain embodiments, the at least one first probe further comprises a 5′ primer-specific portion, wherein the 5′ primer-specific portion comprises a sequence, and the at least one second probe further comprises a 3′ primer-specific portion, wherein the 3′ primer-specific portion comprises a sequence. In certain embodiments, the kit further comprises: at least one primer set, the primer set comprising (i) at least one first primer comprising the sequence of the 5′ primer-specific portion of the first probe, and (ii) at least one second primer comprising a sequence complementary to the sequence of the 3′ primer-specific portion of the second probe.

In certain embodiments, kits for determining the methylation state of a target cytosine in at least one target nucleic acid sequence in a sample comprise: a ligation probe set for each target cytosine, the probe set comprising: (a) at least one first probe, comprising a target-specific portion, and (b) at least one second probe, comprising a target-specific portion, wherein the probes in each set are suitable for ligation together when hybridized adjacent to one another on a complementary target nucleic acid sequence; and a selective ligase that ligates together adjacently hybridized probes only if the target cytosine is unmethylated.

In certain embodiments, the at least one first probe further comprises a 5′ primer-specific portion, wherein the 5′ primer-specific portion comprises a sequence, and the at least one second probe further comprises a 3′ primer-specific portion, wherein the 3′ primer-specific portion comprises a sequence. In certain embodiments, the kit further comprises: at least one primer set, the primer set comprising (i) at least one first primer comprising the sequence of the 5′ primer-specific portion of the first probe, and (ii) at least one second primer comprising a sequence complementary to the sequence of the 3′ primer-specific portion of the second probe.

In certain embodiments, kits comprise one or more additional components, including, without limitation, at least one of: at least one polymerase, at least one transcriptase, at least one ligation agent, oligonucleotide triphosphates, nucleotide analogs, reaction buffers, salts, ions, and stabilizers. In certain embodiments, kits comprise one or more reagents for purifying the ligation products, including, without limitation, at least one of dialysis membranes, chromatographic compounds, supports, and oligonucleotides.

In certain embodiments, the invention also includes reagents useful for performing the methods. Such products can be sold individually or as kits. In certain embodiments, the kits are used to determine the methylation state of cytosines that are believed to have clinical, diagnostic, medical, forensic, or industrial significance.

We claim:

1. A method for determining the methylation state of a target cytosine in at least one target nucleic acid sequence in a sample, comprising:
forming a ligation reaction composition comprising the sample and a ligation probe set for each target nucleic acid sequence, the probe set comprising (a) at least one first probe, comprising a target-specific portion and (b) at least one second probe, comprising a target-specific portion;
subjecting the ligation reaction composition to at least one cycle of ligation, under conditions effective to ligate together first and second probes that are hybridized adjacent to one another on the target nucleic acid sequence only if the target cytosine is methylated or only if the target cytosine is unmethylated, to form a ligation product; and
detecting the presence or absence of the ligation product to determine the methylation state of the target cytosine.

2. The method of claim 1, wherein the at least one first probe comprises a terminal nucleotide that aligns opposite the target cytosine if the at least one first probe is hybridized to the target nucleic acid sequence, and the at least one second probe comprises a terminal nucleotide that aligns opposite a nucleotide that is adjacent to the target cytosine if the at least one second probe is hybridized to the target nucleic acid sequence.

3. The method of claim 1, wherein the detecting comprises separation by a mobility dependent analysis technique.

4. The method of claim 1, wherein at least one of the at least first probe and the at least second probe comprises at least one mobility modifier.

5. The method of claim 1, wherein the at least one cycle of ligation comprises repeated cycles of ligation.

6. The method of claim 1, wherein at least one of the at least one first probe and the at least one second probe is labeled.

7. The method of claim 1, wherein at least one of the at least one first probe and the at least one second probe comprises a tag sequence.

8. The method of claim 1, wherein the at least one first probe comprises a label that has a first detectable signal value when it is ligated to the at least one second probe and has a second detectable signal value when it is not ligated to the at least one second probe.

9. The method of claim 8, wherein the at least one first probe comprises a signal moiety and the at least one second probe comprises a quencher moiety, wherein the quencher moiety changes the detectable signal value from the signal moiety when the at least one first and second probes are ligated together.

10. The method of claim 8, wherein the at least one first probe comprises a signal moiety and the at least one second probe comprises a donor moiety, wherein the donor moiety changes the detectable signal value from the signal moiety when the at least one first and second probes are ligated together.

11. The method of claim 1, wherein at least one of the at least one first probe and the at least one second probe is labeled, and the method further comprises:
after subjecting the ligation reaction composition to at least one cycle of ligation, increasing stringency so that unligated at least one first probe and at least one second probe are not hybridized to the target nucleic acid sequence;
substantially removing any unhybridized at least one first probe and at least one second probe from the sample; and
detecting signal from the label.

12. A method for determining the methylation state of a target cytosine in at least one target nucleic acid sequence in a sample, comprising:
forming a test composition by incubating the at least one target nucleic acid sequence with a modifying agent that converts unmethylated cytosine to a converted nucleotide, but does not convert methylated cytosine to the converted nucleotide, to obtain at least one test target nucleic acid sequence;
forming a ligation reaction composition comprising at least a portion of the test composition and a ligation probe set for each target nucleic acid sequence, the probe set comprising (a) at least one first probe, comprising a target-specific portion and (b) at least one second probe, comprising a target-specific portion, wherein the probes in each probe set are suitable for ligation together when hybridized adjacent to one another on a complementary test target nucleic acid sequence, and wherein at least one of the at least one first probe and the at least one second probe of each probe set comprises a test nucleotide that aligns opposite the target cytosine or the converted nucleotide if the probe is hybridized to the test target nucleic acid sequence, wherein the test nucleotide is complementary to cytosine or to the converted nucleotide;
subjecting the ligation reaction composition to at least one cycle of ligation, wherein adjacently hybridizing probes are ligated together to form a ligation product; and
detecting the presence or absence of the ligation product to determine the methylation state of the target cytosine.

13. The method of claim 12, wherein the test nucleotide is a terminal nucleotide of the at least one first probe, and the at least one second probe comprises a terminal nucleotide that aligns opposite the nucleotide adjacent to the target cytosine or the converted nucleotide if the at least one second probe is hybridized to the test target nucleic acid sequence.

14. The method of claim 12, wherein the detecting comprises separation by a mobility dependent analysis technique.

15. The method of claim 12 wherein at least one of the at least first probe and the at least second probe comprises at least one mobility modifier.

16. The method of claim 12, wherein the at least one cycle of ligation comprises repeated cycles of ligation.

17. The method of claim 12, wherein at least one of the at least one first probe and the at least one second probe is labeled.

18. The method of claim 12, wherein at least one of the at least one first probe and the at least one second probe comprises a tag sequence.

19. The method of claim 12, wherein the at least one first probe comprises a label that has a first detectable signal value when it is ligated to the at least one second probe and has a second detectable signal value when it is not ligated to the at least one second probe.

20. The method of claim 19, wherein the at least one first probe comprises a signal moiety and the at least one second probe comprises a quencher moiety, wherein the quencher moiety changes the detectable signal value from the signal moiety when the at least one first and second probes are ligated together.

21. The method of claim 19, wherein the at least one first probe comprises a signal moiety and the at least one second probe comprises a donor moiety, wherein the donor moiety changes the detectable signal value from the signal moiety when the at least one first and second probes are ligated together.

22. The method of claim 12, wherein at least one of the at least one first probe and the at least one second probe is labeled, and the method further comprises:
after subjecting the ligation reaction composition to at least one cycle of ligation, increasing stringency so that unligated at least one first probe and at least one second probe are not hybridized to the target nucleic acid sequence;
substantially removing any unhybridized at least one first probe and at least one second probe from the sample; and
detecting signal from the label.

23. The method of claim 12, wherein the modifying agent is bisulfite.

24. The method of claim 12, wherein the converted nucleotide is uracil.

25. A method for determining the methylation state of a target cytosine in at least one target nucleic acid sequence in a sample, comprising:
forming a test composition by incubating the at least one target nucleic acid sequence with a modifying agent that modifies unmethylated cytosine, but does not modify methylated cytosine, to obtain at least one test target nucleic acid sequence;
forming a ligation reaction composition comprising at least a portion of the test composition and a ligation probe set for each target nucleic acid sequence, the probe set comprising (a) at least one first probe, comprising a target-specific portion and (b) at least one second probe, comprising a target-specific portion;
subjecting the ligation reaction composition to at least one cycle of ligation, under conditions effective to ligate together first and second probes that are hybridized adjacent to one another on the test target nucleic acid sequence only if the target cytosine is modified or only if the target cytosine is unmodified, to form a ligation product; and
detecting the presence or absence of the ligation product to determine the methylation state of the target cytosine.

26. The method of claim 25, wherein the at least one first probe comprises a terminal nucleotide that aligns opposite the modified or unmodified target cytosine if at least one first probe is hybridized to the target nucleic acid sequence, and the at least one second probe comprises a terminal nucleotide that aligns opposite the nucleotide adjacent to the target modified or unmodified cytosine if the at least one second probe is hybridized to the target nucleic acid sequence.

27. The method of claim 25, wherein the detecting comprises separation by a mobility dependent analysis technique.

28. The method of claim 25, wherein at least one of the at least first probe and the at least second probe comprises at least one mobility modifier.

29. The method of claim 25, wherein the at least one cycle of ligation comprises repeated cycles of ligation.

30. The method of claim 25, wherein at least one of the at least one first probe and the at least one second probe is labeled.

31. The method of claim 25, wherein at least one of the at least one first probe and the at least one second probe comprises a tag sequence.

32. The method of claim 25, wherein the at least one first probe comprises a label that has a first detectable signal value when it is ligated to the at least one second probe and has a second detectable signal value when it is not ligated to the at least one second probe.

33. The method of claim 32, wherein the at least one first probe comprises a signal moiety and the at least one second probe comprises a quencher moiety, wherein the quencher moiety changes the detectable signal value from the signal moiety when the at least one first and second probes are ligated together.

34. The method of claim 32, wherein the at least one first probe comprises a signal moiety and the at least one second probe comprises a donor moiety, wherein the donor moiety changes the detectable signal value from the signal moiety when the at least one first and second probes are ligated together.

35. The method of claim 25, wherein at least one of the at least one first probe and the at least one second probe is labeled, and the method further comprises:
after subjecting the ligation reaction composition to at least one cycle of ligation, increasing stringency so that unligated at least one first probe and at least one second probe are not hybridized to the target nucleic acid sequence;
substantially removing any unhybridized at least one first probe and at least one second probe from the sample; and
detecting signal from the label.

36. A method for determining the methylation state of a target cytosine in at least one target nucleic acid sequence in a sample, comprising:
forming a test composition by incubating the at least one target nucleic acid sequence with a modifying agent that modifies methylated cytosine, but does not modify unmethylated cytosine, to obtain at least one test target nucleic acid sequence;
forming a ligation reaction composition comprising at least a portion of the test composition and a ligation probe set for each target nucleic acid sequence, the probe set comprising (a) at least one first probe, comprising a target-specific portion and (b) at least one second probe, comprising a target-specific portion;
subjecting the ligation reaction composition to at least one cycle of ligation, under conditions effective to ligate together first and second probes that are hybridized adjacent to one another on the test target nucleic acid sequence only if the target cytosine is modified or only if the target cytosine is unmodified, to form a ligation product; and
detecting the presence or absence of the ligation product to determine the methylation state of the target cytosine.

37. The method of claim 36, wherein the at least one first probe comprises a terminal nucleotide that aligns opposite the modified or unmodified target cytosine if at least one first probe is hybridized to the target nucleic acid sequence, and the at least one second probe comprises a terminal nucleotide that aligns opposite the nucleotide adjacent to the target modified or unmodified cytosine if the at least one second probe is hybridized to the target nucleic acid sequence.

38. The method of claim 36, wherein the detecting comprises separation by a mobility dependent analysis technique.

39. The method of claim 36, wherein at least one of the at least first probe and the at least second probe comprises at least one mobility modifier.

40. The method of claim 36, wherein the at least one cycle of ligation comprises repeated cycles of ligation.

41. The method of claim 36, wherein at least one of the at least one first probe and the at least one second probe is labeled.

42. The method of claim 36, wherein at least one of the at least one first probe and the at least one second probe comprises a tag sequence.

43. The method of claim 36, wherein the at least one first probe comprises a label that has a first detectable signal value when it is ligated to the at least one second probe and has a second detectable signal value when it is not ligated to the at least one second probe.

44. The method of claim 43, wherein the at least one first probe comprises a signal moiety and the at least one second probe comprises a quencher moiety, wherein the quencher moiety changes the detectable signal value from the signal moiety when the at least one first and second probes are ligated together.

45. The method of claim 43, wherein the at least one first probe comprises a signal moiety and the at least one second probe comprises a donor moiety, wherein the donor moiety changes the detectable signal value from the signal moiety when the at least one first and second probes are ligated together.

46. The method of claim 36, wherein at least one of the at least one first probe and the at least one second probe is labeled, and the method further comprises:
after subjecting the ligation reaction composition to at least one cycle of ligation, increasing stringency so that unligated at least one first probe and at least one second probe are not hybridized to the target nucleic acid sequence;
substantially removing any unhybridized at least one first probe and at least one second probe from the sample; and
detecting signal from the label.

47. A method for determining the methylation state of a target cytosine in at least one target nucleic acid sequence in a sample, comprising:
forming a ligation reaction composition comprising the sample and a ligation probe set for each target nucleic acid sequence, the probe set comprising (a) at least one first probe, comprising a target-specific portion and a 5′ primer-specific portion, wherein the 5′ primer-specific portion comprises a sequence, and (b) at least one second probe, comprising a target-specific portion and a 3′ primer-specific portion, wherein the 3′ primer-specific portion comprises a sequence;
forming an amplification test composition by subjecting the ligation reaction composition to at least one cycle of ligation, under conditions effective to ligate together first and second probes that are hybridized adjacent to one another on the target nucleic acid sequence only if the target cytosine is methylated or only if the target cytosine is unmethylated, to form a ligation product;
forming an amplification reaction composition comprising:
at least a portion of the amplification test composition;
a polymerase; and
at least one primer set, the primer set comprising (i) at least one first primer comprising the sequence of the 5′ primer-specific portion of the ligation product, and (ii) at least one second primer comprising a sequence complementary to the sequence of the 3′ primer-specific portion of the ligation product;
subjecting the amplification reaction composition to at least one amplification reaction; and
determining the methylation state of the target cytosine by detecting the presence or absence of the ligation product.

48. A method for determining the methylation state of a target cytosine in at least one target nucleic acid sequence in a sample, comprising:
forming a test composition by incubating the at least one target nucleic acid sequence with a modifying agent that converts unmethylated cytosine to a converted nucleotide, but does not convert methylated cytosine to the converted nucleotide, to obtain at least one test target nucleic acid sequence;
forming a ligation reaction composition comprising at least a portion of the test composition and a ligation probe set for each target nucleic acid sequence, the probe set comprising (a) at least one first probe, comprising a target-specific portion and a 5′ primer-specific portion, wherein the 5′ primer-specific portion comprises a sequence, and (b) at least one second probe, comprising a target-specific portion and a 3′ primer-specific portion, wherein the 3′ primer-specific portion comprises a sequence, wherein the probes in each probe set are suitable for ligation together when hybridized adjacent to one another on a complementary test target nucleic acid sequence, and wherein at least one of the at least one first probe and the at least one second probe of each probe set comprises a test nucleotide that aligns opposite the target cytosine or the converted nucleotide if the probe is hybridized to the test target nucleic acid sequence, wherein the test nucleotide is complementary to cytosine or to the converted nucleotide;
forming an amplification test composition by subjecting the ligation reaction composition to at least one cycle of ligation, wherein adjacently hybridizing probes are ligated together to form a ligation product;
forming an amplification reaction composition comprising:
at least a portion of the amplification test composition;
a polymerase; and
at least one primer set, the primer set comprising (i) at least one first primer comprising the sequence of the 5′ primer-specific portion of the ligation product, and (ii) at least one second primer comprising a sequence complementary to the sequence of the 3′ primer-specific portion of the ligation product;
subjecting the amplification reaction composition to at least one amplification reaction; and
determining the methylation state of the target cytosine by detecting the presence or absence of the ligation product.

49. A method for determining the methylation state of a target cytosine in at least one target nucleic acid sequence in a sample, comprising:
forming a test composition by incubating the at least one target nucleic acid sequence with a modifying agent that modifies unmethylated cytosine, but does not modify methylated cytosine, to obtain at least one test target nucleic acid sequence;
forming a ligation reaction composition comprising at least a portion of the test composition and a ligation probe set for each target nucleic acid sequence, the probe set comprising (a) at least one first probe, comprising a target-specific portion and a 5′ primer-specific portion, wherein the 5′ primer-specific portion comprises a sequence, and (b) at least one second probe, comprising a target-specific portion and a 3′ primer-specific portion;
forming an amplification test composition by subjecting the ligation reaction composition to at least one cycle of ligation, under conditions effective to ligate together first and second probes that are hybridized adjacent to one another on the test target nucleic acid sequence only if the target cytosine is modified or only if the target cytosine is unmodified, to form a ligation product;
forming an amplification reaction composition comprising:
at least a portion of the amplification test composition;
a polymerase; and
at least one primer set, the primer set comprising (i) at least one first primer comprising the sequence of the 5′ primer-specific portion of the ligation product, and (ii) at least one second primer comprising a sequence complementary to the sequence of the 3′ primer-specific portion of the ligation product;
subjecting the amplification reaction composition to at least one amplification reaction; and
detecting the presence or absence of the ligation product to determine the methylation state of the target cytosine.

50. A method for determining the methylation state of a target cytosine in at least one target nucleic acid sequence in a sample, comprising:
forming a test composition by incubating the at least one target nucleic acid sequence with a modifying agent that modifies methylated cytosine, but does not modify unmethylated cytosine, to obtain at least one test target nucleic acid sequence;
forming a ligation reaction composition comprising at least a portion of the test composition and a ligation probe set for each target nucleic acid sequence, the probe set comprising (a) at least one first probe, comprising a target-specific portion and a 5′ primer-specific portion, wherein the 5′ primer-specific portion comprises a sequence, and (b) at least one second probe, comprising a target-specific portion and a 3′ primer-specific portion;
forming an amplification test composition by subjecting the ligation reaction composition to at least one cycle of ligation, under conditions effective to ligate together first and second probes that are hybridized adjacent to one another on the test target nucleic acid sequence only if the target cytosine is modified or only if the target cytosine is unmodified, to form a ligation product;
forming an amplification reaction composition comprising:
at least a portion of the amplification test composition;
a polymerase; and
at least one primer set, the primer set comprising (i) at least one first primer comprising the sequence of the 5′ primer-specific portion of the ligation product, and (ii) at least one second primer comprising a sequence complementary to the sequence of the 3′ primer-specific portion of the ligation product;
subjecting the amplification reaction composition to at least one amplification reaction; and
detecting the presence or absence of the ligation product to determine the methylation state of the target cytosine.

51. A kit for determining the methylation state of a target cytosine in at least one target nucleic acid sequence in a sample comprising:
a modifying agent that modifies methylated cytosine, but does not modify unmethylated cytosine; and
a ligation probe set for each target cytosine, the probe set comprising:
(a) at least one first probe, comprising a target-specific portion, and
(b) at least one second probe, comprising a target-specific portion,
wherein the probes in each set are suitable for ligation together when hybridized adjacent to one another on a complementary target nucleic acid sequence.

52. The kit of claim 51, wherein the at least one first probe further comprises a 5′ primer-specific portion, wherein the 5′ primer-specific-portion comprises a sequence, and the at least one second probe further comprises a 3′ primer-specific portion, wherein the 3′ primer-specific portion comprises a sequence, and the kit further comprises:
at least one primer set, the primer set comprising (i) at least one first primer comprising the sequence of the 5′ primer-specific portion of the first probe, and (ii) at least one second primer comprising a sequence complementary to the sequence of the 3′ primer-specific portion of the second probe.

53. A kit for determining the methylation state of a target cytosine in at least one target nucleic acid sequence in a sample comprising:
a modifying agent that modifies unmethylated cytosine, but does not modify methylated cytosine; and;
a ligation probe set for each target cytosine, the probe set comprising:
(a) at least one first probe, comprising a target-specific portion, and
(b) at least one second probe, comprising a target-specific portion,
wherein the probes in each set are suitable for ligation together when hybridized adjacent to one another on a complementary target nucleic acid sequence.

54. The kit of claim 51, wherein the at least one first probe further comprises a 5′ primer-specific portion, wherein the 5′ primer-specific-portion comprises a sequence, and the at least one second probe further comprises a 3′ primer-specific portion, wherein the 3′ primer-specific portion comprises a sequence, and the kit further comprises:
at least one primer set, the primer set comprising (i) at least one first primer comprising the sequence of the 5′ primer-specific portion of the first probe, and (ii) at least one second primer comprising a sequence complementary to the sequence of the 3′ primer-specific portion of the second probe.

55. A kit for determining the methylation state of a target cytosine in at least one target nucleic acid sequence in a sample comprising:
a ligation probe set for each target cytosine, the probe set comprising:
(a) at least one first probe, comprising a target-specific portion, and
(b) at least one second probe, comprising a target-specific portion,
wherein the probes in each set are suitable for ligation together when hybridized adjacent to one another on a complementary target nucleic acid sequence; and
a selective ligase that ligates together adjacently hybridized probes only if the target cytosine is methylated.

56. The kit of claim 55, wherein the at least one first probe further comprises a 5′ primer-specific portion, wherein the 5′ primer-specific portion comprises a sequence, and the at least one second probe further comprises a 3′ primer-specific portion, wherein the 3′ primer-specific portion comprises a sequence, and the kit further comprises:
at least one primer set, the primer set comprising (i) at least one first primer comprising the sequence of the 5′ primer-specific portion of the first probe, and (ii) at least one second primer comprising a sequence complementary to the sequence of the 3′ primer-specific portion of the second probe.

57. A kit for determining the methylation state of a target cytosine in at least one target nucleic acid sequence in a sample comprising:
a ligation probe set for each target cytosine, the probe set comprising:
(a) at least one first probe, comprising a target-specific portion, and
(b) at least one second probe, comprising a target-specific portion,
wherein the probes in each set are suitable for ligation together when hybridized adjacent to one another on a complementary target nucleic acid sequence; and
a selective ligase that ligates together adjacently hybridized probes only if the target cytosine is unmethylated.

58. The kit of claim 57, wherein the at least one first probe further comprises a 5′ primer-specific portion, wherein the 5′ primer-specific portion comprises a sequence, and the at least one second probe further comprises a 3′ primer-specific portion, wherein the 3′ primer-specific portion comprises a sequence, and the kit further comprises:
at least one primer set, the primer set comprising (i) at least one first primer comprising the sequence of the 5′ primer-specific portion of the first probe, and (ii) at least one second primer comprising a sequence complementary to the sequence of the 3′ primer-specific portion of the second probe.
